(19) United States
(12) Patent Application Publication
(10) Pub. No.: US 2009/0246289 A1
(43) Pub. Date: Oct. 1, 2009

(54) SYSTEM AND METHOD FOR DIAGNOSING AND MANAGING THOSE AT RISK FOR CARDIOVASCULAR DISEASE

(76) Inventors: Robert Superko, Marietta, GA (US); Robert Penny, Lebanon, IN (US)

Correspondence Address: SNELL & WILMER L.L.P. (Main), 400 EAST VAN BUREN, ONE ARIZONA CENTER, PHOENIX, AZ, 85004-2202, US

(21) Appl. No.: 12/027,087

(22) Filed: Feb. 6, 2008

Related U.S. Application Data
(60) Provisional application No. 60/888,415, filed on Feb. 6, 2007.

(51) Int. Cl. A61K 35/60; G01N 33/92; A61K 31/397

(52) U.S. Cl. 424/523; 436/71; 514/210.02

(57) ABSTRACT

A system and method for diagnosing and managing patients at risk for cardiovascular disease. The system and method provides for automating the ordering of a number of advanced tests, compiling and evaluating test results, and producing an automated report which includes recommended disease management options.

### CROSS-REFERENCE TO RELATED APPLICATIONS

This application claims priority to provisional patent application having Ser. No. 60/888,415, filed Feb. 6, 2007, which is herein incorporated in its entirety.

### FIELD OF INVENTION

The present invention is directed to a system and method for diagnosing and managing patients at risk for cardiovascular disease. The method includes performing a number of advanced tests to assist in evaluating the lipid and metabolic risks associated with cardiovascular disease and then managing a patient's risk of the disease by recommending various treatment options based on the test results of the patient. The system and method of the present invention translates the latest clinical research into usable treatment options which are based on the test results of patients.

### BACKGROUND OF THE INVENTION

Close to 5 million people suffer from congestive heart failure in the United States and approximately 550,000 new cases present themselves every year. Many people fail to experience symptoms, such as with left ventricular dysfunction, and as a result their disease remains undiagnosed.

The leading cause of morbidity and mortality in the United States is coronary artery disease. Using a single criterion such as total cholesterol levels in different population groups to determine the risk of heart attack and stroke has had limited success. In fact, it has been found that many cases of coronary artery disease occur in people with normal levels of cholesterol.

Currently, no system, method or tool exists which applies comprehensive testing with the latest clinical research and scientifically referenced clinical pathways in order to determine and manage those at risk for cardiovascular disease. Accordingly, there is a need for a system and method which combines advanced lipid and metabolic testing to evaluate cardiovascular disease risk and help manage that risk.

### SUMMARY OF THE INVENTION

The present invention is directed to a system and method for diagnosing and managing patients at risk for cardiovascular disease. The system and method include performing a number of advanced tests to assist in evaluating the lipid and metabolic risks associated with cardiovascular disease and then managing a patient's risk of the disease by recommending various treatment options based on the test results of the patient.

The advanced tests may include, but are not limited to, brain natriuretic peptide (BNP), C-reactive protein, total cholesterol, high density lipoprotein cholesterol (HDL-C), the ratio of cholesterol to HDL-C, triglycerides, calculated low density lipoprotein cholesterol (LDL-C), fibrinogen, homocysteine, low density lipoprotein (LDL) subfractions including LDL 1, LDL 2, LDL 3, LDL 4, LDL 5, LDL 6, LDL 7, calculated LDL, calculated mean LDL particle size, lipoprotein(a), Apo E genotype, plasminogin activator inhibitor 1 (PAI-1), insulin, and pro-BNP. Various treatment options may include, but are not limited to, diet recommendations, exercise recommendations, medications, and additional testing.

Test results and recommendation may be provided in the form of a report which facilitates the presentation and summary of information and recommendations. The system and method of the present invention are intended to be carried out with computer software and hardware.

### BRIEF DESCRIPTION OF THE DRAWINGS

FIGS. 1-14 illustrate flowcharts of exemplary embodiments of the system and method of the present invention for diagnosing and managing patients at risk for cardiovascular disease.

FIGS. 15-16 show an exemplary patient report generated in accordance with the system and method of the present invention for diagnosing and managing patients at risk for cardiovascular disease.

FIGS. 17-19 show another exemplary patient report generated in accordance with the system and method of the present invention for diagnosing and managing patients at risk for cardiovascular disease.

### DETAILED DESCRIPTION

The detailed description of exemplary embodiments herein makes reference to the accompanying drawings and pictures, which show the exemplary embodiment by way of illustration and its best mode. While these exemplary embodiments are described in sufficient detail to enable those skilled in the art to practice the invention, it should be understood that other embodiments may be realized and that logical and mechanical changes may be made without departing from the spirit and scope of the invention. Thus, the detailed description herein is presented for purposes of illustration only and not of limitation. For example, the steps recited in any of the method or process descriptions may be executed in any order and are not limited to the order presented. Moreover, any of the functions or steps may be outsourced to or performed by one or more third parties. Furthermore, any reference to singular includes plural embodiments, and any reference to more than one component may include a singular embodiment.

For the sake of brevity, conventional data networking, application development and other functional aspects of the systems (and components of the individual operating components of the systems) may not be described in detail herein. Furthermore, the connecting lines shown in the various figures contained herein are intended to represent exemplary functional relationships and/or physical couplings between the various elements. It should be noted that many alternative or additional functional relationships or physical connections may be present in a practical system.

The various system components discussed herein may include one or more of the following: a host server or other computing systems including a processor for processing digital data; a memory coupled to the processor for storing digital data; an input digitizer coupled to the processor for inputting digital data; an application program stored in the memory and accessible by the processor for directing processing of digital data by the processor; a display device coupled to the processor and memory for displaying information derived from digital data processed by the processor; and a plurality of databases. Various databases used herein may include: patient data such as family history, demography and environmental data, biological sample data, prior treatment and protocol data, patient clinical data, test data including but not limited to patient or individual data on brain natriuretic peptide (BNP), C-reactive protein, total cholesterol, high density lipoprotein cholesterol (HDL-C), the ratio of cholesterol to HDL-C, triglycerides, calculated low density lipoprotein cholesterol (LDL-C), fibrinogen, homocysteine, low density lipoprotein (LDL) subfractions including LDL 1, LDL 2, LDL 3, LDL 4, LDL 5, LDL 6, LDL 7, calculated LDL, calculated mean LDL particle size, lipoprotein(a), Apo E genotype, plasminogin activator inhibitor 1 (PAI-1), insulin, and pro-BNP, disease management options, patient tracking data, file management data, financial management data, billing data and/or like data useful in the operation of the system. As those skilled in the art will appreciate, user computer may include an operating system (e.g., Windows NT, 95/98/2000, OS2, UNIX, Linux, Solaris, MacOS, etc.) as well as various conventional support software and drivers typically associated with computers. The computer may include any suitable personal computer, network computer, workstation, minicomputer, mainframe or the like. User computer can be in a home or medical/business environment with access to a network. In an exemplary embodiment, access is through a network or the Internet through a commercially-available web-browser software package.

As used herein, the term “network” shall include any electronic communications means which incorporates both hardware and software components of such. Communication among the parties may be accomplished through any suitable communication channels, such as, for example, a telephone network, an extranet, an intranet, Internet, point of interaction device, personal digital assistant (e.g., Palm Pilot®, Blackberry®), cellular phone, kiosk, etc.), online communications, satellite communications, off-line communications, wireless communications, transponder communications, local area network (LAN), wide area network (WAN), networked or linked devices, keyboard, mouse and/or any suitable communication or data input modality. Moreover, although the system is frequently described herein as being implemented with TC P/IP communications protocols, the system may also be implemented using IPX, Appletalk, IP-6, NetBIOS, OSI or any number of existing or future protocols. If the network is in the nature of a public network, such as the Internet, it may be advantageous to presume the network to be insecure and open to eavesdroppers. Specific information related to the protocols, standards, and application software utilized in connection with the Internet is generally known to those skilled in the art and, as such, need not be detailed herein. See, for example, DILIP NAIK, INTERNET STANDARDS AND PROTOCOLS (1998); JAVA 2 COMPLETE, various authors, (Sybex 1999); DEBORAH RAY AND ERIC RAY, MASTERING HTML 4.0 (1997); and LOSHIN, TC P/IP CLEARLY EXPLAINED (1997) and DAVID GOURLEY AND BRIAN TOTTY, HTTP, THE DEFINITIVE GUIDE (2002), the contents of which are hereby incorporated by reference.

The various system components may be independently, separately or collectively suitably coupled to the network via data links which includes, for example, a connection to an Internet Service Provider (ISP) over the local loop as is typically used in connection with standard modem communication, cable modem, Dish networks, ISDN, Digital Subscriber Line (DSL), or various wireless communication methods, see, e.g., GILBERT HELD, UNDERSTANDING DATA COMMUNICATIONS (1996), which is hereby incorporated by reference. It is noted that the network may be implemented as other types of networks, such as an interactive television (ITV) network. Moreover, the system contemplates the use, sale or distribution of any goods, services or information over any network having similar functionality described herein.

As used herein, “transmit” may include sending electronic data from one system component to another over a network connection. Additionally, as used herein, “data” may include encompassing information such as commands, queries, files, data for storage, and the like in digital or any other form.

The system contemplates uses in association with web services, utility computing, pervasive and individualized computing, security and identity solutions, autonomic computing, commodity computing, mobility and wireless solutions, open source, biometrics, grid computing and/or mesh computing.

Any databases discussed herein may include relational, hierarchical, graphical, or object-oriented structure and/or any other database configurations. Common database products that may be used to implement the databases include DB2 by IBM (White Plains, N.Y.), various database products available from Oracle Corporation (Redwood Shores, Calif.), Microsoft Access or Microsoft SQL Server by Microsoft Corporation (Redmond, Wash.), or any other suitable database product. Moreover, the databases may be organized in any suitable manner, for example, as data tables or lookup tables. Each record may be a single file, a series of files, a linked series of data fields or any other data structure. Association of certain data may be accomplished through any desired data association technique such as those known or practiced in the art. For example, the association may be accomplished either manually or automatically. Automatic association techniques may include, for example, a database search, a database merge, GREP, AGREP, SQL, using a key field in the tables to speed searches, sequential searches through all the tables and files, sorting records in the file according to a known order to simplify lookup, and/or the like. The association step may be accomplished by a database merge function, for example, using a “key field” in pre-selected databases or data sectors.

More particularly, a “key field” partitions the database according to the high-level class of objects defined by the key field. For example, certain types of data may be designated as a key field in a plurality of related data tables and the data tables may then be linked on the basis of the type of data in the key field. The data corresponding to the key field in each of the linked data tables is preferably the same or of the same type. However, data tables having similar, though not identical, data in the key fields may also be linked by using AGREP, for example. In accordance with one embodiment, any suitable data storage technique may be utilized to store data without a standard format. Data sets may be stored using any suitable technique, including, for example, storing individual files using an ISO/IEC 7816-4 file structure; implementing a domain whereby a dedicated file is selected that exposes one or more elementary files containing one or more data sets; using data sets stored in individual files using a hierarchical filing system; data sets stored as records in a single file (including compression, SQL accessible, hashed via one or more keys, numeric, alphabetical by first tuple, etc.); Binary Large Object (BLOB); stored as ungrouped data elements encoded using ISO/IEC 7816-6 data elements; stored as ungrouped data elements encoded using ISO/IEC Abstract Syntax Notation (ASN 1) as in ISO/IEC 8824 and 8825; and/or other proprietary techniques that may include fractal compression methods, image compression methods, etc.

In one exemplary embodiment, the ability to store a wide variety of information in different formats is facilitated by storing the information as a BLOB. Thus, any binary information can be stored in a storage space associated with a data set. The BLOB method may store data sets as ungrouped data elements formatted as a block of binary via a fixed memory offset using either fixed storage allocation, circular queue techniques, or best practices with respect to memory management (e.g., paged memory, least recently used, etc.). By using BLOB methods, the ability to store various data sets that have different formats facilitates the storage of data by multiple and unrelated owners of the data sets. For example, a first data set which may be stored may be provided by a first party, a second data set which may be stored may be provided by an unrelated second party, and yet a third data set which may be stored, may be provided by a third party unrelated to the first and second party. Each of these three exemplary data sets may contain different information that is stored using different data storage formats and/or techniques. Further, each data set may contain subsets of data that also may be distinct from other subsets.

As stated above, in various embodiments, the data can be stored without regard to a common format. However, in one exemplary embodiment, the data set (e.g., BLOB) may be annotated in a standard manner when provided for manipulating the data. The annotation may comprise a short header, trailer, or other appropriate indicator related to each data set that is configured to convey information useful in managing the various data sets. For example, the annotation may be called a “condition header”, “header”, “trailer”, or “status”, herein, and may comprise an indication of the status of the data set or may include an identifier correlated to a specific issuer or owner of the data. Subsequent bytes of data may be used to indicate for example, the identity of the issuer or owner of the data, user, transaction/membership account identifier or the like. Each of these condition annotations are further discussed herein.

The data set annotation may also be used for other types of status information as well as various other purposes. For example, the data set annotation may include security information establishing access levels. The access levels may, for example, be configured to permit only certain individuals, levels of employees, companies, or other entities to access data sets, or to permit access to specific data sets based on the transaction, issuer or owner of data, user or the like. Furthermore, the security information may restrict/permit only certain actions such as accessing, modifying, and/or deleting data sets. In one example, the data set annotation indicates that only the data set owner or the user are permitted to delete a data set, various identified users may be permitted to access the data set for reading, and others are altogether excluded from accessing the data set. However, other access restriction parameters may also be used allowing various entities to access a data set with various permission levels as appropriate. The data, including the header or trailer may be received by a stand alone interaction device configured to add, delete, modify, or augment the data in accordance with the header or trailer.

One skilled in the art will also appreciate that, for security reasons, any databases, systems, devices, servers or other components of the system may consist of any combination thereof at a single location or at multiple locations, wherein each database or system includes any of various suitable security features, such as firewalls, access codes, encryption, decryption, compression, decompression, and/or the like.

The computing unit of the web client may be further equipped with an Internet browser connected to the Internet or an intranet using standard dial-up, cable, DSL or any other Internet protocol known in the art. Transactions originating at a web client may pass through a firewall in order to prevent unauthorized access from users of other networks. Further, additional firewalls may be deployed between the varying components of CMS to further enhance security.

Firewall may include any hardware and/or software suitably configured to protect CMS components and/or enterprise computing resources from users of other networks. Further, a firewall may be configured to limit or restrict access to various systems and components behind the firewall for web clients connecting through a web server. Firewall may reside in varying configurations including Stateful Inspection, Proxy based and Packet Filtering among others. Firewall may be integrated within an web server or any other CMS components or may further reside as a separate entity.

The computers discussed herein may provide a suitable website or other Internet-based graphical user interface which is accessible by users. In one embodiment, the Microsoft Internet Information Server (IIS), Microsoft Transaction Server (MTS), and Microsoft SQL Server, are used in conjunction with the Microsoft operating system, Microsoft NT web server software, a Microsoft SQL Server database system, and a Microsoft Commerce Server. Additionally, components such as Access or Microsoft SQL Server, Oracle, Sybase, Informix MYSQL, Interbase, etc., may be used to provide an Active Data Object (ADO) compliant database management system.

Any of the communications, inputs, storage, databases or displays discussed herein may be facilitated through a website having web pages. The term “web page” as it is used herein is not meant to limit the type of documents and applications that might be used to interact with the user. For example, a typical website might include, in addition to standard HTML documents, various forms, Java applets, JavaScript, active server pages (ASP), common gateway interface scripts (CGI), extensible markup language (XML), dynamic HTML, cascading style sheets (CSS), helper applications, plug-ins, and the like. A server may include a web service that receives a request from a web server, the request including a URL and an IP address. The web server retrieves the appropriate web pages and sends the data or applications for the web pages to the IP address. Web services are applications that are capable of interacting with other applications over a communications means, such as the internet. Web services are typically based on standards or protocols such as XML, XSLT, SOAP, WSDL and UDDI. Web services methods are well known in the art, and are covered in many standard texts. See, e.g., ALEX NGHIEM, IT WEB SERVICES: A ROAD MAP FOR THE ENTERPRISE (2003), hereby incorporated by reference.

The clinical database (which may be web-based) for the system and method of the present invention preferably has the ability to upload and store clinical data files in native formats and is searchable on any clinical parameter. The database is also scalable and may utilize an EAV data model (metadata) to enter clinical annotations from any study for easy integration with other studies. In addition, the web-based clinical database is flexible and may be XML and XSLT enabled to be able to add user customized questions dynamically. Further, the database includes exportability to CDISC OD M.

Practitioners will also appreciate that there are a number of methods for displaying data within a browser-based document. Data may be represented as standard text or within a fixed list, scrollable list, drop-down list, editable text field, fixed text field, pop-up window, and the like. Likewise, there are a number of methods available for modifying data in a web page such as, for example, free text entry using a keyboard, selection of menu items, check boxes, option boxes, and the like.

The system and method may be described herein in terms of functional block components, flow charts, screen shots, optional selections and various processing steps. It should be appreciated that such functional blocks may be realized by any number of hardware and/or software components configured to perform the specified functions. For example, the system may employ various integrated circuit components, e.g., memory elements, processing elements, logic elements, look-up tables, and the like, which may carry out a variety of functions under the control of one or more microprocessors or other control devices. Similarly, the software elements of the system may be implemented with any programming or scripting language such as C, C++, Macromedia Cold Fusion, Microsoft Active Server Pages, Java, COBOL, assembler, PERL, Visual Basic, SQL Stored Procedures, extensible markup language (XML), with the various algorithms being implemented with any combination of data structures, objects, processes, routines or other programming elements. Further, it should be noted that the system may employ any number of conventional techniques for data transmission, signaling, data processing, network control, and the like. Still further, the system could be used to detect or prevent security issues with a client-side scripting language, such as JavaScript, VBScript or the like. For a basic introduction of cryptography and network security, see any of the following references: (1) “Applied Cryptography: Protocols, Algorithms, And Source Code In C,” by Bruce Schneier, published by John Wiley & Sons (second edition, 1995); (2) “Java Cryptography” by Jonathan Knudson, published by O'Reilly & Associates (1998); (3) “Cryptography & Network Security: Principles & Practice” by William Stallings, published by Prentice Hall; all of which are hereby incorporated by reference.

As used herein, the term “end user”, “consumer”, “customer”, “client”, “treating physician”, “hospital”, or “business” may be used interchangeably with each other, and each shall mean any person, entity, machine, hardware, software or business. Each participant is equipped with a computing device in order to interact with the system and facilitate online data access and data input. The customer has a computing unit in the form of a personal computer, although other types of computing units may be used including laptops, notebooks, hand held computers, set-top boxes, cellular telephones, touch-tone telephones and the like. The owner/operator of the system and method of the present invention has a computing unit implemented in the form of a computer-server, although other implementations are contemplated by the system including a computing center shown as a main frame computer, a mini-computer, a PC server, a network of computers located in the same of different geographic locations, or the like. Moreover, the system contemplates the use, sale or distribution of any goods, services or information over any network having similar functionality described herein.

In one exemplary embodiment, each client customer may be issued an “account” or “account number”. As used herein, the account or account number may include any device, code, number, letter, symbol, digital certificate, smart chip, digital signal, analog signal, biometric or other identifier/indicia suitably configured to allow the consumer to access, interact with or communicate with the system (e.g., one or more of an authorization/access code, personal identification number (PIN), Internet code, other identification code, and/or the like). The account number may optionally be located on or associated with a charge card, credit card, debit card, prepaid card, embossed card, smart card, magnetic stripe card, bar code card, transponder, radio frequency card or an associated account. The system may include or interface with any of the foregoing cards or devices, or a fob having a transponder and RFID reader in RF communication with the fob. Although the system may include a fob embodiment, the invention is not to be so limited. Indeed, system may include any device having a transponder which is configured to communicate with RFID reader via RF communication. Typical devices may include, for example, a key ring, tag, card, cell phone, wristwatch or any such form capable of being presented for interrogation. Moreover, the system, computing unit or device discussed herein may include a “pervasive computing device,” which may include a traditionally non-computerized device that is embedded with a computing unit. The account number may be distributed and stored in any form of plastic, electronic, magnetic, radio frequency, wireless, audio and/or optical device capable of transmitting or downloading data from itself to a second device.

As will be appreciated by one of ordinary skill in the art, the system may be embodied as a customization of an existing system, an add-on product, upgraded software, a stand alone system, a distributed system, a method, a data processing system, a device for data processing, and/or a computer program product. Accordingly, the system may take the form of an entirely software embodiment, an entirely hardware embodiment, or an embodiment combining aspects of both software and hardware. Furthermore, the system may take the form of a computer program product on a computer-readable storage medium having computer-readable program code means embodied in the storage medium. Any suitable computer-readable storage medium may be utilized, including hard disks, CD-ROM, optical storage devices, magnetic storage devices, and/or the like.

The system and method is described herein with reference to screen shots, block diagrams and flowchart illustrations of methods, apparatus (e.g., systems), and computer program products according to various embodiments. It will be understood that each functional block of the block diagrams and the flowchart illustrations, and combinations of functional blocks in the block diagrams and flowchart illustrations, respectively, can be implemented by computer program instructions.

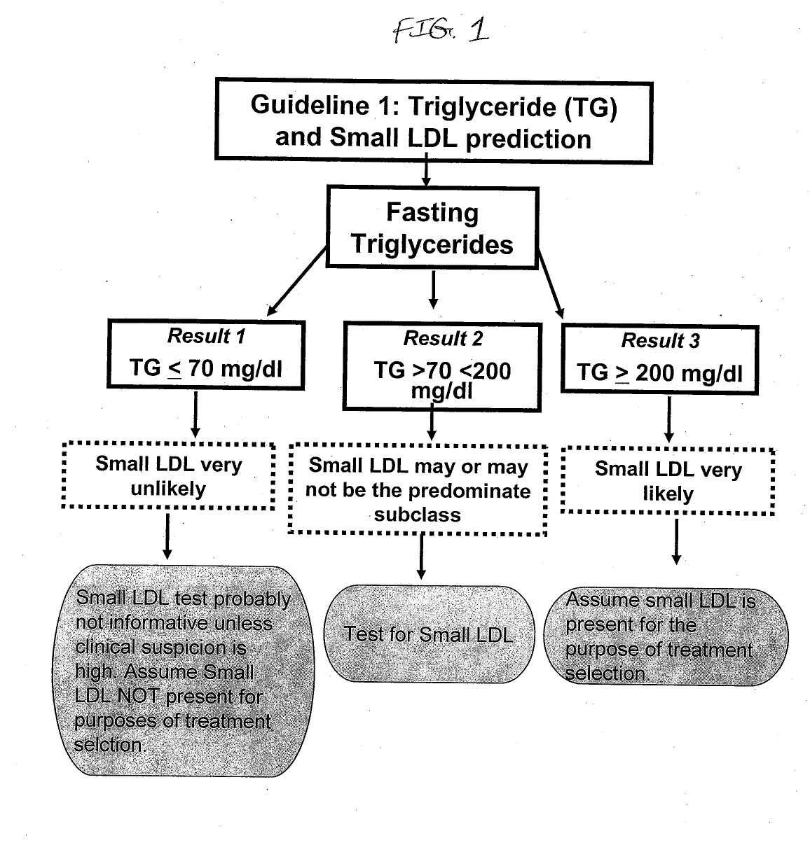

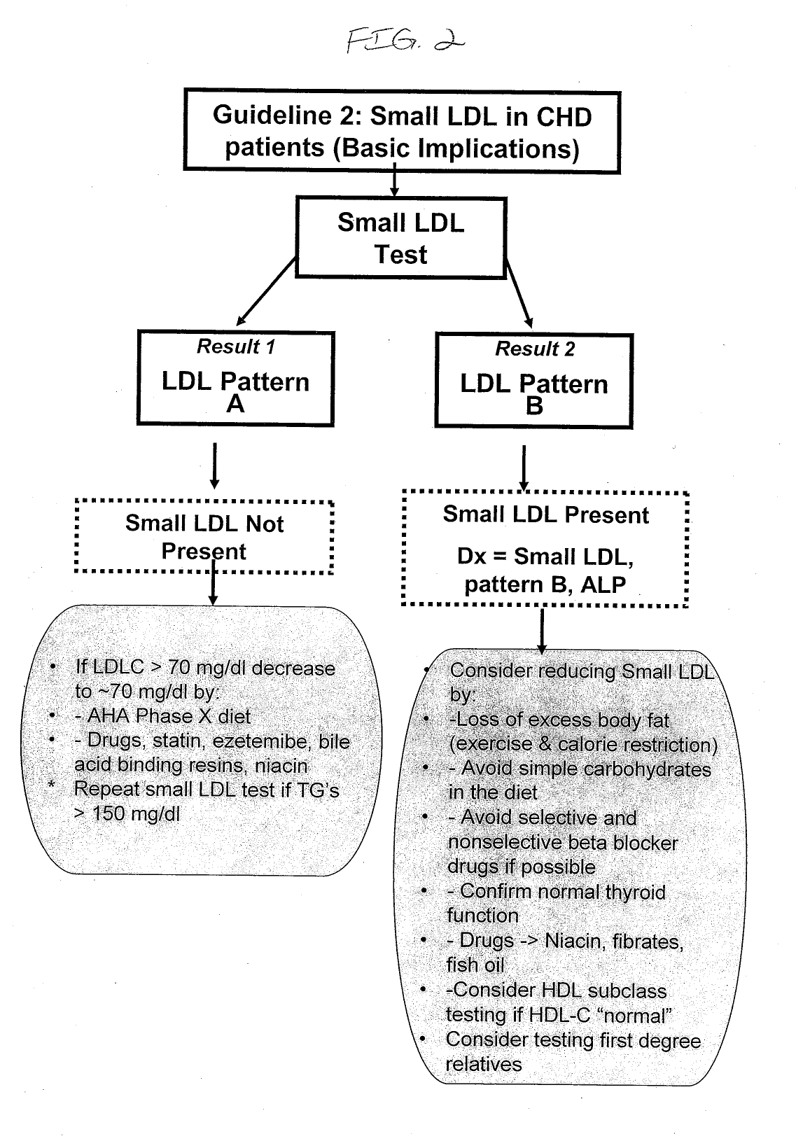

Guidelines 1 through 14 shown in FIGS. 1-14 illustrate the system and method of the present invention for diagnosing and managing patients at risk for cardiovascular disease. The 14 guidelines, along with associated tests performed with the guidelines, background of the tests, and results of those tests are described in the following pages of specification. Testing, compilation of test results, decisions and recommendations for further testing, and recommendations for disease management options may all be automated utilizing computer hardware and software including web-based applications. Patient reports reflecting the tests, test results, and recommendations may also be provided and these reports may also be automated.

Guideline #1. Fasting Triglyceride Values and Small LDL testing in CHD Patients

Statements Regarding Diagnosis and Management are Suggestions Only and Medical Decision Making Must be Individualized to the Patient.

### Test

Fasting Triglyceride (TG)

### Background

A statistically significant inverse relationship exists (r˜0.55, p<0.001) between fasting triglycerides and LDL peak particle diameter (PPD). In general, the higher the TG value, the small the LDL size. However, there is much overlap within the physiologic range of 70-200 mg/dl. FIG. 20 represents the scatter plot between fasting TG and LDL PPD in patients with established CHD.

Blood TG levels can vary a considerable amount in any one patient on a day-to-day basis. For this reason 2-3 TG values obtained on different days may be helpful when the patient's TG value is in the borderline range for clinical decision making. Confirm that the TO value represents a true 12-hour fasting value.

FIG. 1 illustrates a flowchart for carrying out a first guideline in the system and method of the present invention for diagnosing and managing patients at risk for cardiovascular disease.

Result #1. TG≦70 mg/dl
Indicates that LDL particles are very unlikely to be predominantly small and dense and the atherogenic lipoprotein profile (ALP) is probably not being expressed.
The small LDL test probably will not be informative.
Assume small LDL is not predominantly present for purposes of treatment decisions unless other clinical suspicion is present.
Monitor triglyceride (TG) levels. Consider small LDL test if TG≧150 mg/dl. Change in medical statis and lifestyle such as diet, body fat, exercise may impact fasting TG levels. Some medications and OTC agents may impact TG levels.
Search for other metabolic disorders contributing to CHD risk since small LDL is an unlikely contributor at the time of this test.
   Result #2. TG>70<200 mg/dl
Indicates that LDL particles may or may not be predominantly small and dense.
Testing for the small LDL trait may be informative in this group.
Test for the small LDL trait if it contributes to risk determination.
Test for the small LDL trait if it contributes to treatment decisions.
Test for the small LDL trait if useful as a baseline to assess lifestyle or medication affect.
Test for the small LDL trait if it contributes to family counseling.
Monitor TG levels. Increases in TG often reflect increased small LDL presence and reduction in TG often reflect reduced small LDL.
If HDL-C>40 mg/dl in men and >50 mg/dl in premenopausal women (or postmenopausal women on HRT) consider HDL subclass determination in order to detect abnormalities in HDL subclass distribution not reflected by HDL-C value.
   Result #3. TG≧200 mg/dl
Indicates that LDL particles are very likely to be predominantly small and dense and the atherogenic lipoprotein profile (ALP) is probably being expressed.
The small LDL test probably will not be informative.
Assume small LDL is predominantly present for purposes of treatment decisions unless other clinical suspicion is present.
Monitor triglyceride (TG) levels. Consider small LDL test if TG<200 mg/dl since LDL subclass phenotype may have changed.

### RELATED READING HEREIN INCORPORATED BY REFERENCE

1. Austin M A. Triglyceride, small, dense low-density lipoprotein, and the atherogenic lipoprotein phenotype. Curr Atheroscler Rep 2000 May; 2(3):200-7.
2. Austin M A, King M C, Vranizan K M, Krauss R M. Atherogenic lipoprotein phenotype. A proposed genetic marker for coronary heart disease risk. Circulation 1990; 82:495-506.
3. Superko H R. Hypercholesterolemia and dyslipidemia. Current Treatment Options in Cardiovascular Medicine 2000; 2:173-187.
4. Krauss R M. Atherogenicity of triglyceride-rich lipoproteins. Am J Cardiol 1998; 81:13B-17B.

### Guideline #2. LDL Subclass Distribution in CHD Patients (Basic Implications)

### Test

LDL subclass pattern test

### Background

LDL is not a homogeneous category of lipoproteins, but consists of a set of discrete subspecies with distinct molecular properties. In normal subjects, four to seven major LDL subspecies, distinguished by size and density, can be identified. Often; but not always, associated with elevations in plasma triglycerides is the dense LDL subclass pattern, (LDL pattern B), which is a heritable trait determined by a single major dominant gene (the ALP locus). The gene has been designated ATHS (for atherosclerosis susceptibility) and located on the short arm of chromosome 19, 0.5 CM from the LDL receptor. Based on Hardy-Weinberg equilibrium, 30-35% of people are heterozygous for ALP and another 5% are homozygous. The dense LDL subspecies increases CAD risk 3-fold and risk increases substantially when the small LDL trait is present in combination with other risk factors such as elevated Apo B and elevated insulin.

FIG. 2 illustrates a flowchart for carrying out a second guideline in the system and method of the present invention for diagnosing and managing patients at risk for cardiovascular disease.

### Result #1. LDL Pattern A.

Indicates that. LDL particles are not predominantly small and dense and the atherogenic lipoprotein profile (ALP) is not being expressed.
If LDL-C is elevated reduce LDL-C to ˜70 mg/dl through
   Diet: AHA Step II low-fat diet and consider
   Drugs: Statin+ezetemibe or +bile acid binding resin. Nicotinic Acid (NA) may also be helpful in reducing LDLC. Monitor and titrate doses and safety tests as required.
Monitor triglyceride (TG) levels. Repeat small LDL test if TG 50 mg/dl. Elevation in fasting TG levels may reflect expression of the small LDL trait and affected by body fat, carbohydrates in the diet, exercise levels, some medications, and some medical conditions.
Search for other metabolic disorders contributing to CHD risk since small LDL is not a contributor at the time of this test.

### Result #2. LDL Pattern B.

Indicates that LDL particles are predominantly small and dense, and reflects the presence of ALP. This indicates a 3-fold increased risk for CHD in an asymptomatic population and in a CHD population, a 2-fold increased rate of arteriographic progression compared to patients with a predominance of large LDL particles. ALP is associated with insulin resistance and reflects a high risk for the future development of type 2 diabetes. ALP has many of the characteristics of what is now termed the “Metabolic Syndrome”.
Reduce small, dense LDL and LDL-C to 70 mg/dl through
   Lifestyle: Loss of excess body fat, optimization of exercise.
   Diet: Avoid simple carbohydrates, alcohol.
   Drugs: Avoid selective and non-selective beta-blockers if medically appropriate. Consider treatment with NA, fibrate, NA plus fibrate, or NA plus statin. Fish oil supplementation may be helpful. Monitor and titrate therapy as required.
Monitor TG levels. Increases in TG often reflect increased small LDL presence and reduction in TG often reflect reduced small LDL.
Consider measurement of LDL Apo B as a determination of LDL particle number.
If HDL-C>40<65 mg/dl in men and >50<75 mg/dl in premenopausal women (or postmenopausal women on HRT) consider HDL subclass determination in order to detect abnormalities in HDL subclass distribution not reflected by HDL-C value.
Consider family screening. The small LDL trait is inherited and screening first degree relatives for fasting triglycerides and the small LDL trait if appropriate may be informative.

### RELATED READING INCORPORATED BY REFERENCE

1. Krauss R. Is the size of low-density lipoprotein particles related to the risk of coronary heart disease? JAMA 2002; 287:712-713.
2. Austin M A, Mykkanen L, Kuusisto J, Edwards K L, Nelson C, Haffner S M, Pyorala K, Laakso M. Prospective study of small LDLs as a risk factor for non-insulin dependent diabetes mellitus in elderly men and women. Circulation 1995; 92:1770-1778.
3. Krauss R M, Burke D J. Identification of multiple subclasses of plasma low density lipoproteins in normal humans. J Lipid Res 1982; 23:97-104.
4. Austin M A, King M C, Vranizan K M, Krauss R M. Atherogenic lipoprotein phenotype. A proposed genetic marker for coronary heart disease risk. Circulation 1990; 82:495-506.
5. Nishina P M, Johnson J P, Naggert J K, Krauss R M. Linkage of atherogenic lipoprotein phenotype to the low-density lipoprotein receptor locus on the short arm of chromosome 19. Proc Natl Acad Sci USA 1992; 89:708-712.
6. Lamarche B, Tchemof A, Mauriege P, Cantin B, Dagenais G R, Lupien P J, Despres J P. Fasting insulin and apolipoprotein B levels and low-density lipoprotein particle size as risk factors for ischemic heart disease. JAMA 1998; 279:1955-1961.
7. Sniderman A D, Cianflone K. Measurement of apoproteins: time to improve the diagnosis and treatment of the atherogenic dyslipoproteinemias. Clin Chem. 1996; 42:489-491.
   Guideline #3: LDL-C 70-130 mg/dl and LDL Pattern A

Statements Regarding Diagnosis and Management are Suggestions Only and Medical Decision Making Must be Individualized to the Patient.

### Test

LDL subclass test

### Background

The small LDL trait is not present and in patients with CHD a focus on LDL-C reduction and search for other metabolic disorders may be productive. Safely achieving a LDL-C close to 70 mg/dl appears to reduce CHD risk.

Consider testing for Lp(a), HDL subclass abnormalities, homocysteine, and hyperfibrinogenemia since the LDL-C is not dramatically elevated.

If triglyceride (TG) levels increases, consider retesting for small LDL to check for adverse effects on LDL subclass distribution.
Consider AHA reduced fat or mediteranean diet to assist LDL-C reduction.
Consider a moderately powerful statin to assist LDL-C reduction.
Consider ezetimibe±statin to assist LDL-C reduction.
Consider a low-dose bile-acid-binding resin to assist LDL-C reduction.
Measure baseline apo B for subsequent assessment of change in LDL particle number and screening for hyperapobetalipoproteinemia.

### Repeat Standard Lipid Profile (TG, TC, LDL-C, HDL-C)

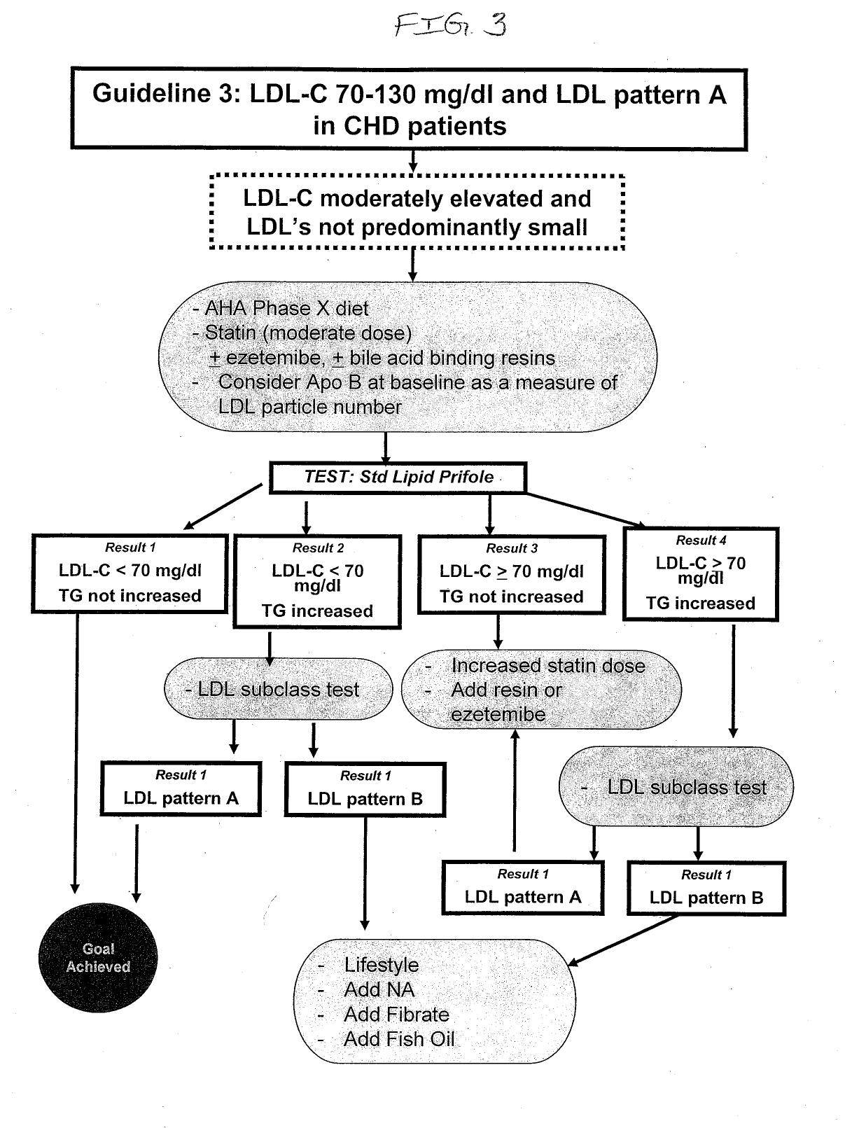

FIG. 3 illustrates a flowchart for carrying out a third guideline in the system and method of the present invention for diagnosing and managing patients at risk for cardiovascular disease.

Result #1. LDL-C<70 mg/dl, TG has not Increased.

Goal achieved.

Monitor standard lipid profile and safety tests.

Result #2. LDL-C<70 mg/dl, TG has Increased.

Repeat the LDL subclass test:
If LDL pattern A, the goal is achieved.
If LDL pattern B:
Avoid simple carbohydrates in the diet
Achieve optimal body weight
Consider increased physical activity
Consider the use of NA, fibrates, or fish oil.
   Result #3. LDL-C≧70 mg/dl, TG has not Increased.

Consider increasing statin dose, switching to a more powerful statin, or adding ezetimibe or a BABR to reduce LDL-C to <70 mg/dl.
Monitor standard lipid profile and safety tests.
   Result #4. LDL-C≧70 mg/dl, TG has Increased.

Repeat the LDL subclass test:
If LDL pattern A, consider increasing statin dose, switching to a more powerful statin, or adding ezetimibe or a BABR to reduce LDL-C to <70 mg/dl.
If LDL pattern B:
Avoid simple carbohydrates in the diet
Achieve optimal body weight
Consider increased physical activity
Consider the use of NA, fibrates, or fish oil.

### RELATED READING INCORPORATED BY REFERENCE

1. NCEP ATP-III. Expert Panel on Detection, Evaluation, and Treatment of High Blood Cholesterol in Adults. Executive Summary of The Third Report of The National Cholesterol Education Program (NCEP) Expert Panel on Detection, Evaluation, And Treatment of High Blood Cholesterol In Adults (Adult Treatment Panel III). JAMA 2001; 285:2486-2497.
2. LaRosa J C, Grundy S M, Waters D D, Shear C, Barter P, Fruchart J C, Gotto A M, Greten H, Kastelein J J, Shepherd J, Wenger N K; Treating to New Targets (TNT) Investigators. Intensive lipid lowering with atorvastatin in patients with stable coronary disease. N Engl J. Med. 2005 Apr. 7; 352(14):1425-35. Epub 2005 Mar. 8.
3. Cannon C P, Braunwald E, McCabe C H, Rader D J, Rouleau J L, Belder R, Joyal S V, Hill K A, Pfeffer M A, Skene A M; Pravastatin or Atorvastatin Evaluation and Infection Therapy-Thrombolysis in Myocardial Infarction 22 Investigators. Intensive versus moderate lipid lowering with statins after acute coronary syndromes. N Engl J. Med. 2004 Apr. 8; 350(15):1495-504. Epub 2004 Mar. 8.
4. H. Robert Superko. The Metabolic Syndrome and Coronary Artery Disease. Hurst's The Heart 2004 McGraw-Hill, New York, edited by: V. Fuster et al. Pp 2127-2141.
5. Williams P T, Superko H R, Haskell W L, Alderman E A, Blanche P J, Holl L G, Krauss R M. Smallest LDL particles are most strongly related to coronary disease progression in Men. Arterioscler Thromb Vasc Biol. 2003; 23:314-321.
6. H. Robert Superko, M D, Mark. E. McGovern, M D, Elaine Raul, Brenda Garrett, R N. Nicotinic Acid has a Differential Effect on Low Density Lipoprotein Subclass Distribution in Patients Classified as LDL pattern A, B, or I. Am J Card 2004; 94:588-594.
7. Superko H R, Berneis, K K, Williams, P T, Rizzo, M, Wood, P D. Differential Effect of Gemfibrozil in Normolipemic Subjects with Predominantly Dense or Buoyant Low Density Lipoprotein Particles and the Effect on Postprandial Lipemia and Lp(a). Am J Cardiology 2005; 96: 1266-1272.
8. Khan et al. JLR 2002; 43:979-985.
9. Maki et al. J Am Coll Nutr 2005; 24: 189-199.

10. O'Keefe J H Jr et al. Effects of pravastatin with NA or magnesium on lipid levels and postprandial lipemia. Am J. Cardiol. 1995 Sep. 1; 76(7):480-484.

Guideline #4: LDL-C 70-130 mg/dl and LDL Pattern B

### Statements Regarding Diagnosis and Management are Suggestions Only and Medical Decision Making Must be Individualized to the Patient.

### Test

LDL Subclass Test

### Background

The small LDL trait is present in combination with moderately elevated LDL-C. In patients with CHD this combination often requires combination drug therapy in addition to lifestyle modification. Safely achieving a LDL-C close to 70 mg/dl appears to reduce CHD risk (X). The presence of small LDL implies an increased rate of arteriographic progression and increased CHD event risk (x,x). Disordered reverse cholesterol transport is often present in these patients as well. The presence of small LDL amplifies the risk of other risk factors such as elevated Apo B and elevated insulin (x).
Avoid simple carbohydrates in the diet and consider a mediteranean diet.
Reduce excess body fat (x).
Consider a moderately powerful statin to assist LDL-C reduction.
Consider NA to assist in small LDL and Apo B reduction.
Consider a fibrate if NA is not tolerated or contraindicated.
Consider fish oil supplement.
Consider ezetimibe+statin to assist LDL-C reduction.
Consider a low-dose bile-acid-binding resin to assist LDL-C reduction.
Measure baseline apo B for subsequent assessment of change in LDL particle number.

### Repeat Standard Lipid Profile (TG, TC, LDL-C, HDL-C)

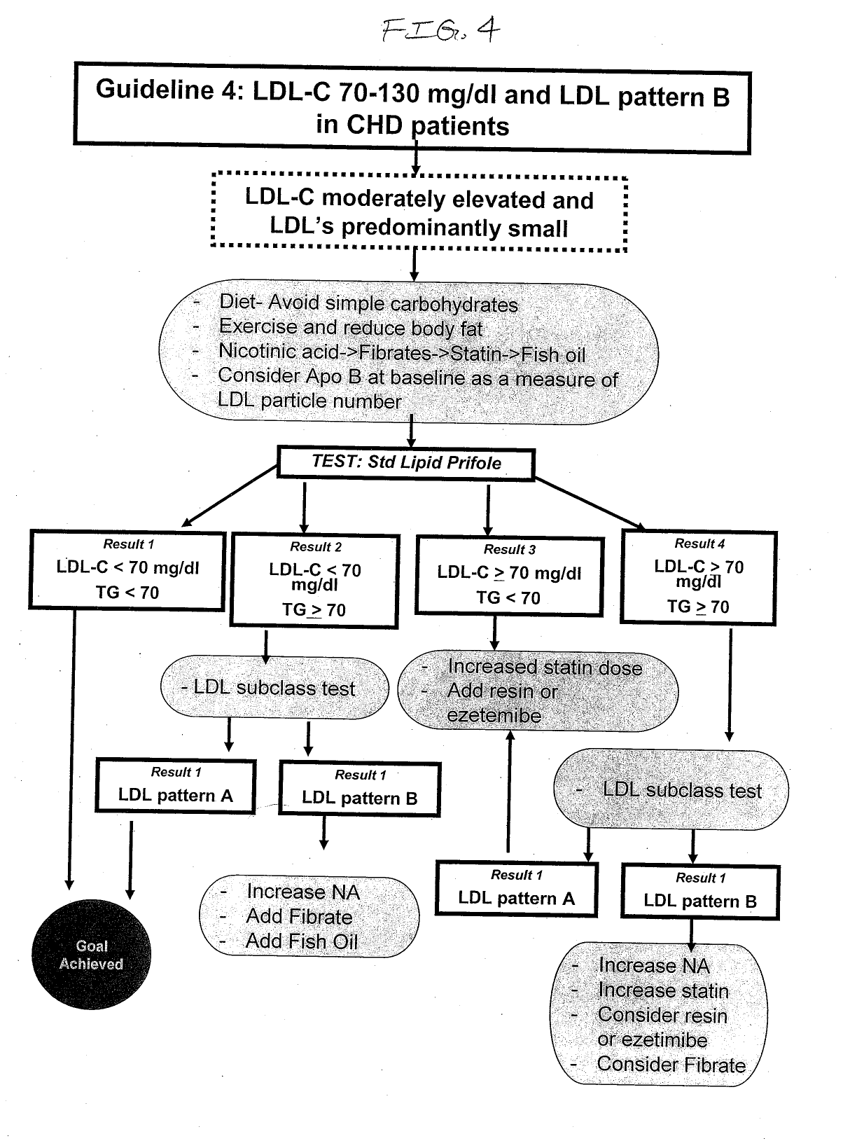

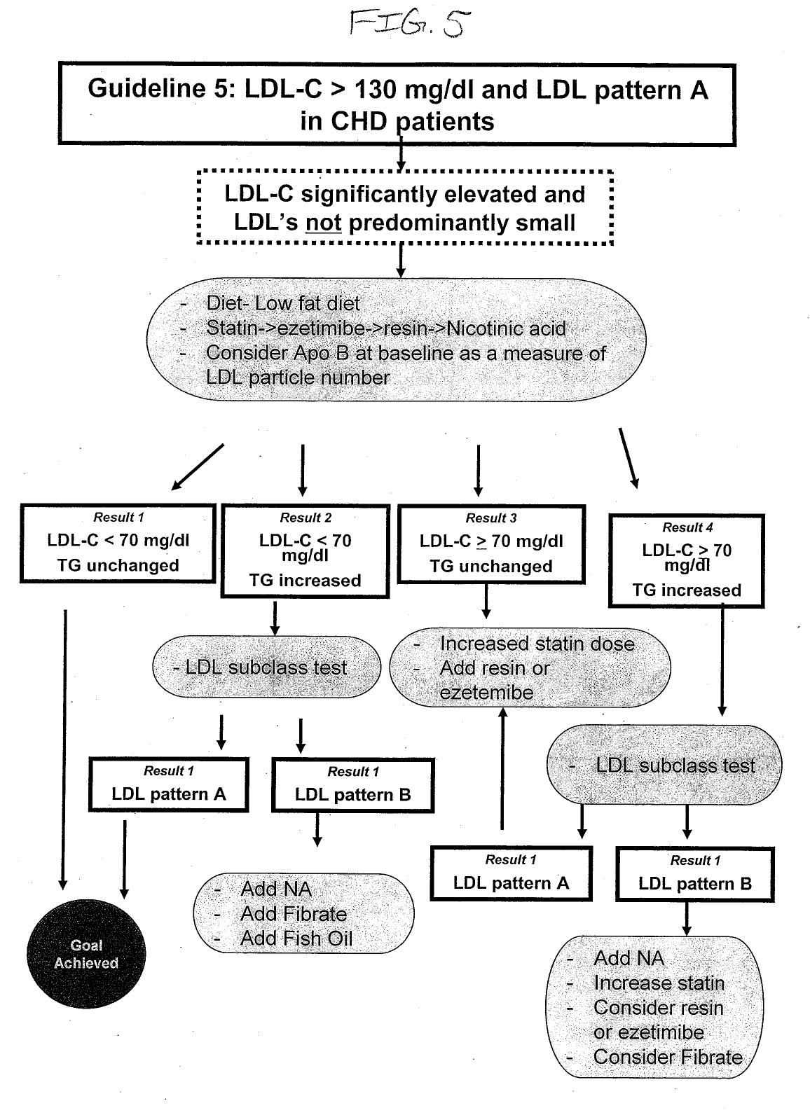

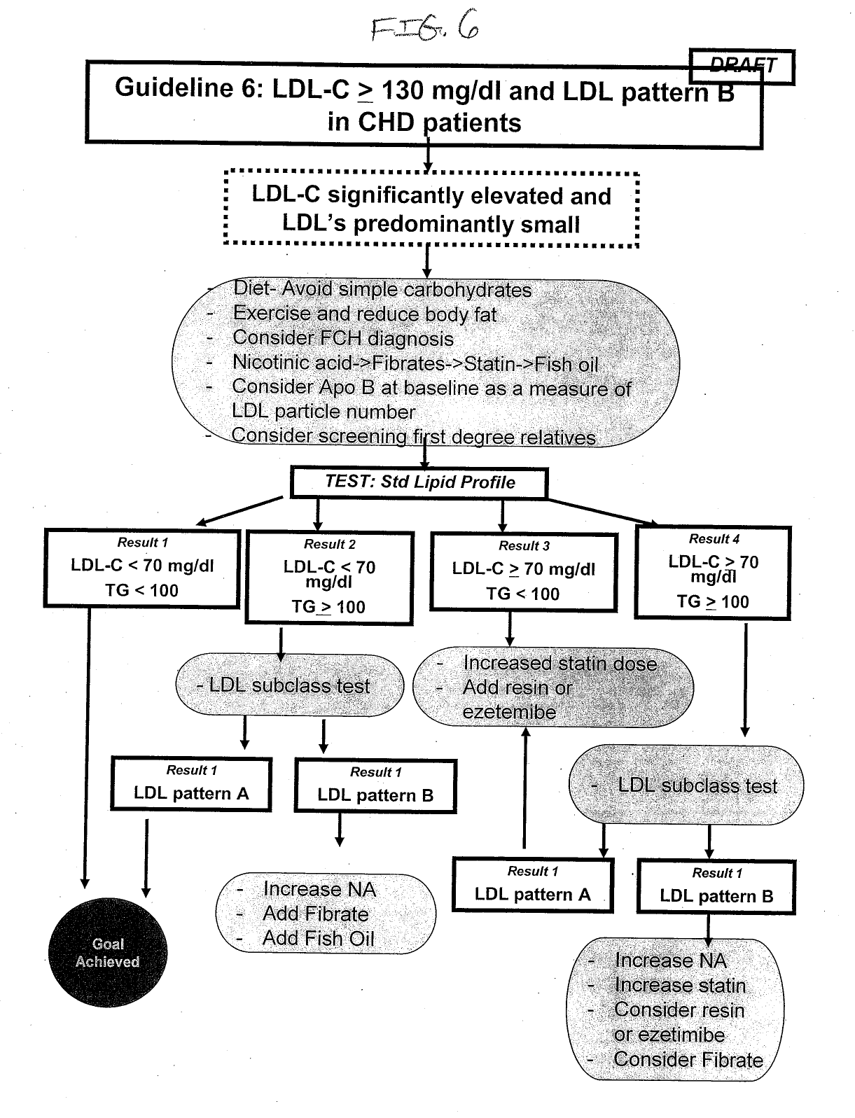

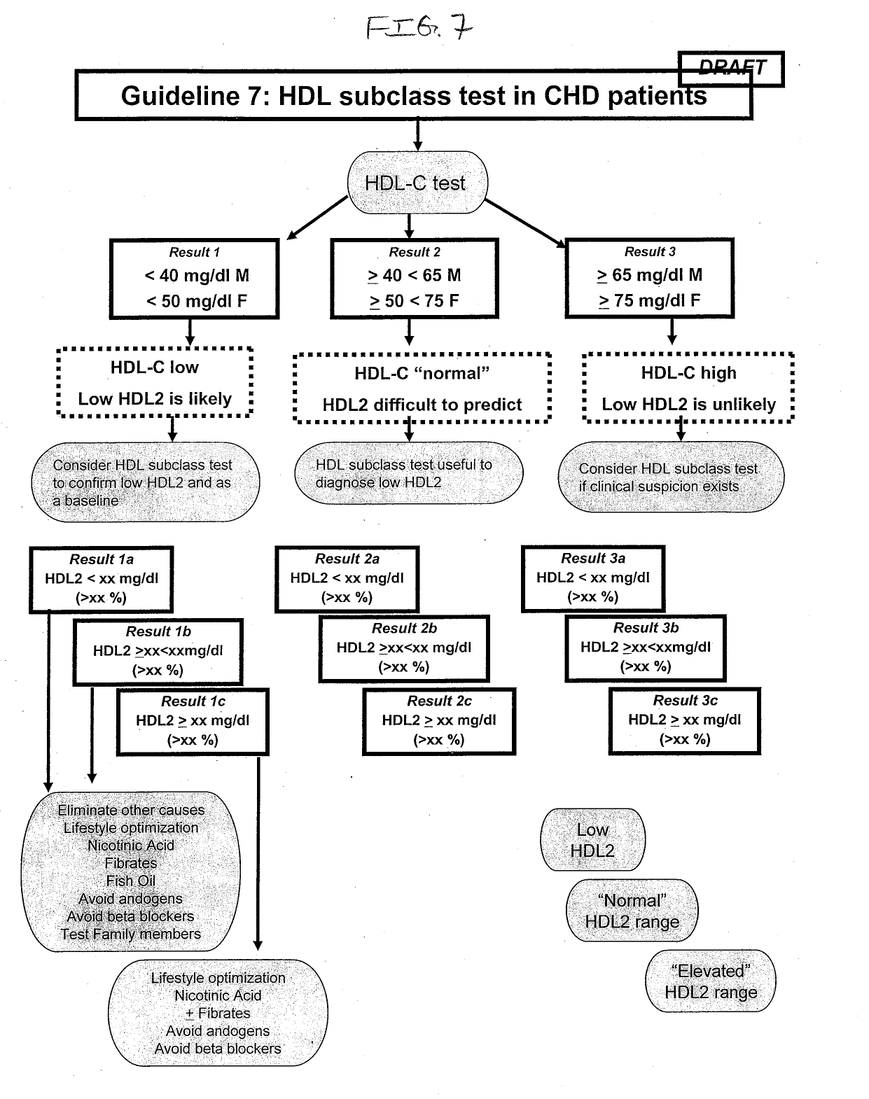

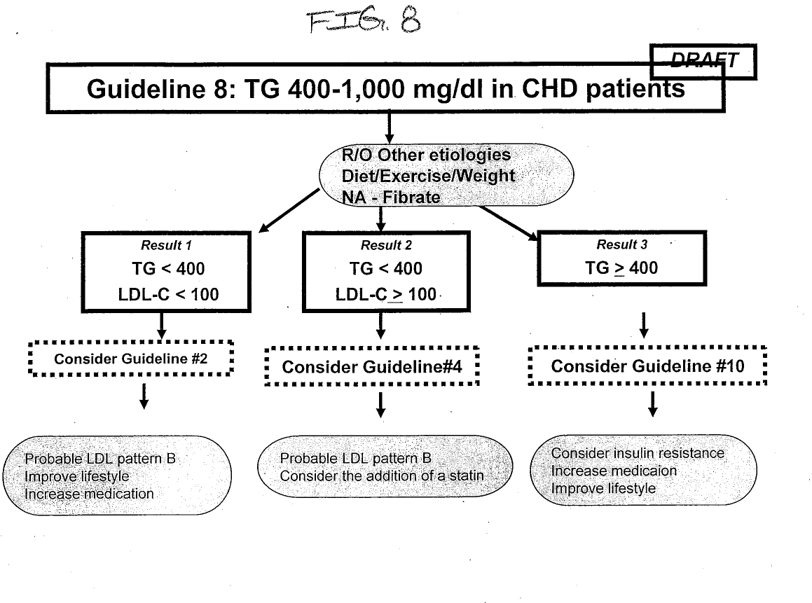

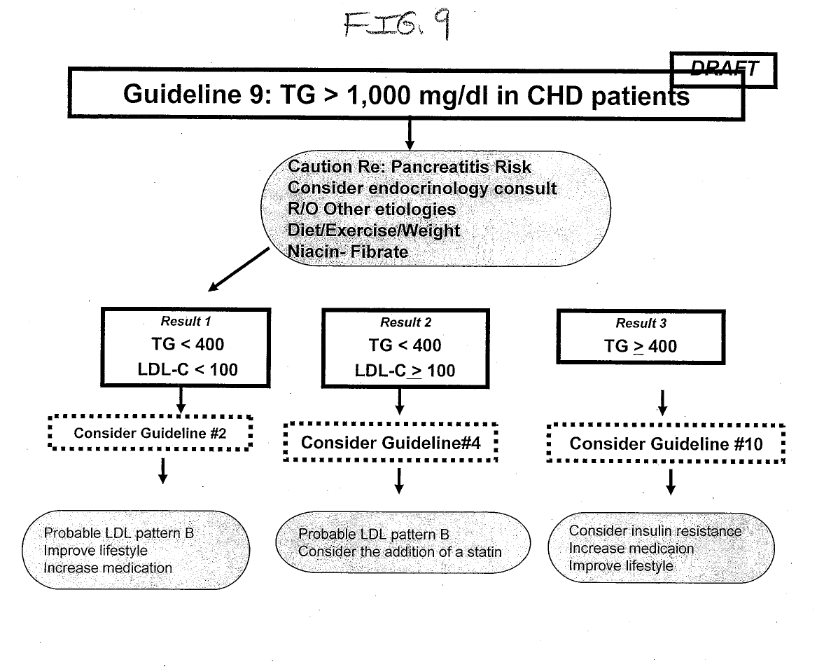

FIG. 4 illustrates a flowchart for carrying out a fourth guideline in the system and method of the present invention for diagnosing and managing patients at risk for cardiovascular disease.

Result #1. LDL-C<70 mg/dl, TG<70 mg/dl.

Goal achieved.

Monitor standard lipid profile and safety tests.

Result #2. LDL-C<70 mg/dl, TG≧70 mg/dl.

Repeat LDL subclass test
If LDL pattern A, the goal is achieved.
If LDL pattern B:
   Optimize lifestyle
   Consider titrating NA dose to a higher level.
   Consider adding a fibrate if medically acceptable.
   Consider adding a fish oil supplement.
   Monitor for potential drug interactions.
      Result #3. LDL-C≧70 mg/dl and TG<70 mg/dl.
Consider increasing the statin dose or switching to a more powerful statin.
Consider ezetimibe+statin to assist LDL-C reduction.
Consider a low-dose bile-acid-binding resin to assist LDL-C reduction.
   Result #4. LDL-C≧70 mg/dl and TG≧70 mg/dl.

Repeat LDL subclass test
If LDL pattern A:
Consider increasing the statin dose or switching to a more powerful statin.
Consider ezetimibe+statin to assist LDL-C reduction.
Consider a low-dose bile-acid-binding resin to assist LDL-C reduction.
If LDL pattern B:
Optimize lifestyle
Consider titrating NA dose to a higher level.
Consider adding a fibrate if medically acceptable.
Consider increasing the statin dose or switching to a more powerful statin.
Consider ezetimibe+statin to assist LDL-C reduction.
Consider a low-dose bile-acid-binding resin to assist LDL-C reduction.
Consider adding a fish oil supplement.
Monitor for potential drug interactions.

### RELATED READING INCORPORATED BY REFERENCE

1. Dreon D M, Fernstrom H A, Williams P T, Krauss R M. LDL subclass patterns and lipoprotein response to a low-fat, high-carbohydrate diet in women. Arterioscler Thromb VascBiol. 1997; 17:707-714.
2. Superko H R, Krauss R M. Differential effects of nicotinic acid in subjects with different LDL subclass patterns. Atherosclerosis 1992; 95:69-76.
3. Superko H R, Haskell W L, Krauss R M. Association of lipoprotein subclass distribution with use of selective and non-selective beta-blocker medications in patients with coronary heart disease. Atherosclerosis. 1993; 101: 1-8.
4. Superko H R, Wood P D, Krauss R M. Effect of alpha- and selective beta-blockade for hypertension control on plasma lipoproteins, apoproteins, lipoprotein subclasses, and postprandial lipemia. Am J Med 1989; 86:26-31.
5. Superko H R. Hypercholesterolemia and dyslipidemia. Current Treatment Options in Cardiovascular Medicine 2000; 2:173-187.
6. Krauss R M, Blanche P J. Detection and quantitation of LDL subfractions. Curr Opin Lipidol 1992:3:377-383.
7. Superko H R. Small, Dense LDL Subclass Pattern B: Issues for Clinicians. Current Atherosclerosis Reports. 1999; 1:50-57.
8. Minihane A M, Khan S, Leigh-Firbank E C, et al. ApoE polymorphism and fish oil supplementation in subjects with an atherogenic lipoprotein phenotype. Arterioscler Thromb Vasc Biol. 2000; 20:1990-1997.
9. Sniderman A D, Cianflone K. Measurement of apoproteins: time to improve the diagnosis and treatment of the atherogenic dyslipoproteinemias. Clin Chem. 1996; 42:489-491.
   Guideline #5: LDL-C≧130 mg/dl and LDL pattern A

### Statements Regarding Diagnosis and Management are Suggestions Only and Medical Decision Making Must be Individualized to the Patient.

### Test

LDL-C
LDL subclass test

### Background

When the small LDL trait is not present in patients with CHD, a focus on LDL-C reduction and a search for other metabolic disorders may be productive. Elevated LDL-C levels likely play an atherogenic role in patients of this type. Safely achieving a LDL-C close to 70 mg/dl appears to reduce CHD risk.

Consider testing for Lp(a), HDL subclass abnormalities, homocysteine, and hyperfibrinogenemia since the risk of these disorders is exacerbated by elevated LDL-C levels. If triglyceride (TG) levels increase a clinically significant amount, consider retesting for small LDL to check for adverse effects on LDL subclass distribution.
Consider AHA reduced fat or Mediterranean diet to assist LDL-C reduction.
Consider a moderately powerful statin to assist LDL-C reduction.
Consider ezetimibe+statin to assist LDL-C reduction.
Consider a low-dose bile-acid-binding resin+statin to assist LDL-C reduction.
The combination of a statin+nicotinic acid has been used successfully in familial heterozygous hyperlipidemia patients.
Measure baseline apo B for subsequent assessment of change in LDL particle number.

### Repeat Standard Lipid Profile (TG, TC, LDL-C, HDL-C)

FIG. 5 illustrates a flowchart for carrying out a fifth guideline in the system and method of the present invention for diagnosing and managing patients at risk for cardiovascular disease.

Result #1. LDL-C<70 mg/dl and TG has not Increased.

Goal achieved.

Monitor standard lipid profile and safety tests.

Result #2. LDL-C<70 mg/dl and TG has Increased an Amount Felt to be Clinically Significant.

Repeat the LDL subclass test:
If LDL pattern A, the goal is achieved.
If LDL pattern B:
Avoid simple carbohydrates in the diet.
Achieve optimal body weight.
Consider increased physical activity.
Consider the use of NA, fibrates, or fish oil.
Be aware of beta blocker (selective and nonselective) effects on TG, small LDL, and HDL2.
   Result #3. LDL-C≧70 mg/dl and TG has not increased.

Consider increasing statin dose, switching to a more powerful statin, or adding ezetimibe or a BABR to reduce LDL-C to <70 mg/dl.
Monitor standard lipid profile and safety tests.
   Result #4. LDL-C≧70 mg/dl and TG has Increased.

Repeat the LDL subclass test:
If LDL pattern A, consider increasing statin dose, switching to a more powerful statin, or adding ezetimibe, or a BABR, or nicotinic acid to reduce LDL-C to <70 mg/dl.
If LDL pattern B:
Avoid simple carbohydrates in the diet.
Achieve optimal body weight.
Consider increased physical activity.
Consider the use of NA, fibrates, or fish oil.
Be aware of beta blocker (selective and nonselective) effects on TG, small LDL, and HDL2.

### RELATED READING INCORPORATED BY REFERENCE

1. NCEP ATP-III. Expert Panel on Detection, Evaluation, and Treatment of High Blood Cholesterol in Adults. Executive Summary of The Third Report of The National Cholesterol Education Program (NCEP) Expert Panel on Detection, Evaluation, And Treatment of High Blood Cholesterol In Adults (Adult Treatment Panel III). JAMA 2001; 285:2486-2497.
2. LaRosa J C, Grundy S M, Waters D D, Shear C, Barter P, Fruchart J C, Gotto A M, Greten H, Kastelein J J, Shepherd J, Wenger N K; Treating to New Targets (TNT) Investigators. Intensive lipid lowering with atorvastatin in patients with stable coronary disease. N Engl J. Med. 2005 Apr. 7; 352(14):1425-35. Epub 2005 Mar. 8.
3. Kane J P, Malloy M J, Ports T A, Phillips N R, Diehl J C, Havel R J. Regression of coronary atherosclerosis during treatment of familial hypercholesterolemia with combined drug regimens. JAMA 1990; 264:3007-3012.
4. Cannon C P, Braunwald E, McCabe C H, Rader D J, Rouleau J L, Belder R, Joyal S V, Hill K A, Pfeffer M A, Skene A M; Pravastatin or Atorvastatin Evaluation and Infection Therapy-Thrombolysis in Myocardial Infarction 22 Investigators. Intensive versus moderate lipid lowering with statins after acute coronary syndromes. N Engl J. Med. 2004 Apr. 8; 350(15):1495-504. Epub 2004 Mar. 8.
5. Williams P T, Superko H R, Haskell W L, Alderman E A, Blanche P J, Holl L G, Krauss R M. Smallest LDL particles are most strongly related to coronary disease progression in Men. Arterioscler Thromb Vasc Biol. 2003; 23:314-321.
6. H. Robert Superko, M D, Mark E. McGovern, M D, Elaine Raul, Brenda Garrett, R N. Nicotinic Acid has a Differential Effect on Low Density Lipoprotein Subclass Distribution in Patients Classified as LDL pattern A, B, or I. Am J Card 2004; 94:588-594.
7. Superko H R, Berneis, K K, Williams, P T, Rizzo, M, Wood, P D. Differential Effect of Gemfibrozil in Normolipemic Subjects with Predominantly Dense or Buoyant Low Density Lipoprotein Particles and the Effect on Postprandial Lipemia and Lp(a). Am J Cardiology 2005; 96:1266-1272.
8. Khan S, Minihane A M, Talmud P J, Wright J W, Murphy M C, Williams C M, Griffin B A. Dietary long-chain n-3 PUFAs increase LPL gene expression in adipose tissue of subjects with an atherogenic lipoprotein phenotype. J Lipid Res. 2002 June; 43(6):979-85.
9. Maki K C, Van Elswyk M E, McCarthy D, Hess S P, Veith P E, Bell M, Subbaiah P, Davidson M H. Lipid responses to a dietary docosahexaenoic acid supplement in men and women with below average levels of high density lipoprotein cholesterol. J Am Coll Nutr. 2005 June; 24(3):189-99.
10. Krauss R M. Genetic, Metabolic, and Dietary Influences on the Atherogenic Lipoprotein Phenotype. Simopoulos AP (ed): Geneitc Variation and Dietary Response. World Rev Nutr Diet. Basel, Karger 1997; 80:22-43.

### Guideline #6: LDL-C>130 MG/DL and LDL Pattern B

### Statements Regarding Diagnosis and Management are Suggestions Only and Medical Decision Making Must be Individualized to the Patient.

### Test

LDL-C
LDL subclass test

### Background

The small LDL trait is present in combination with significantly elevated LDL-C. Consider the diagnosis of Familial Combined Hyperlipidemia (FCH). The diagnostic criteria for FCH have recently been modified to include a fasting TG>133 mg/dl and apo B>120 mg/dl. A diagnosis of FCH implies a 4-fold increased CHD risk along with inherited attributes and family implications. In patients with CHD this combination often requires combination drug therapy in addition to lifestyle modification. Safely achieving a LDL-C close to 70 mg/dl appears to reduce CHD risk. The presence of small LDL implies an increased rate of arteriographic progression and increased CHD event risk. Disordered reverse cholesterol transport is often present in these patients as well and identified by reduced HDL-C and low HDL2. The presence of small LDL amplifies the risk of other risk factors such as elevated Apo B and elevated insulin. CHD patients with this disorder can benefit greatly from treatment with a Nicotinic Acid+statin, or Nicotinic Acid+bile acid binding resin combination.
Rule-out primary and secondary causes of hyperlipidemia.
Avoid simple carbohydrates in the diet and consider a Mediterranean diet.
Reduce excess body fat.
Consider a moderately powerful statin to assist LDL-C reduction.
Consider Nicotinic Acid to assist in small LDL and Apo B reduction.
Consider a fibrate if Nicotinic Acid is not tolerated or contraindicated.
Consider a fish oil supplement.
Consider ezetimibe+statin to assist LDL-C reduction.
Consider a bile-acid-binding resin+statin to assist LDL-C reduction.
Measure baseline apo B for subsequent assessment of change in LDL particle number and to aid in the diagnosis of FCH.
Consider screening first-degree family members.

### Repeat Standard Lipid Profile (TG, TC, LDL-C, HDL-C)

FIG. 6 illustrates a flowchart for carrying out a sixth guideline in the system and method of the present invention for diagnosing and managing patients at risk for cardiovascular disease.

Result #1. LDL-C<70 mg/dl and TG<100 mg/dl.

Goal achieved.

Monitor standard lipid profile and safety tests.

Result #2. LDL-C<70 mg/dl and TG≧100 mg/dl.

Repeat LDL subclass test
If LDL pattern A, the goal is achieved.
If LDL pattern B:
Optimize lifestyle.
Consider titrating Nicotine Acid dose to a higher level.
Consider adding a fibrate if medically acceptable.
Consider adding a fish oil supplement.
Monitor for potential drug interactions.
   Result #3. LDL-C≧70 mg/dl and TG<100 mg/dl.
Consider increasing the statin dose or switching to a more powerful statin
Consider ezetimibe+statin to assist LDL-C reduction.
Consider a low-dose bile-acid-binding resin to assist LDL-C reduction.
   Result #4. LDL-C≧70 mg/dl and TG≧100 mg/dl.

Repeat LDL subclass test
If LDL pattern A:
Consider increasing the statin dose or switching to a more powerful statin.
Consider ezetimibe+statin to assist LDL-C reduction.
Consider a low-dose bile-acid-binding resin to assist LDL-C reduction.
If LDL pattern B:
Optimize lifestyle.
Consider titrating Nicotic Acid dose to a higher level.
Consider adding a fibrate if medically acceptable.
Consider increasing the statin dose or switching to a more powerful statin.
Consider ezetimibe+statin to assist LDL-C reduction.
Consider a low-dose bile-acid-binding resin to assist LDL-C reduction.
Consider the combination of Nicotinic Acid+Statin+bile-acid-binding resin.
Consider adding a fish oil supplement.
Monitor for potential drug interactions

### RELATED READING INCORPORATED BY REFERENCE

1. de Graaf J, van der Vleuten G, Stalenhoef A F. Diagnostic criteria in relation to the pathogenesis of familial combined hyperlipidemia. Semin Vasc Med. 2004 August; 4(3):229-40.
2. Cannon C P, Braunwald E, McCabe C H, Rader D J, Rouleau J L, Belder R, Joyal S V, Hill K A, Pfeffer M A, Skene A M; Pravastatin or Atorvastatin Evaluation and Infection Therapy-Thrombolysis in Myocardial Infarction 22 Investigators. Intensive versus moderate lipid lowering with statins after acute coronary syndromes. N Engl J. Med. 2004 Apr. 8; 350(15):1495-504.
3. Lamarche B, Tchernof A, Mauriege P, Cantin B, Dagenais G R, Lupien P J, Despres J P. Fasting insulin and apolipoprotein B levels and low-density lipoprotein particle size as risk factors for ischemic heart disease. JAMA 1998; 279:1955-1961.
4. Dreon D M, Fernstrom H A, Williams P T, Krauss R M. LDL subclass patterns and lipoprotein response to a low-fat, high-carbohydrate diet in women. Arterioscler Thromb Vasc Biol. 1997; 17:707-714.
5. Superko H R, Krauss R M. Differential effects of nicotinic acid in subjects with different LDL subclass patterns. Atherosclerosis 1992; 95:69-76.
6. Superko H R, Haskell W L, Krauss R M. Association of lipoprotein subclass distribution with use of selective and non-selective beta-blocker medications in patients with coronary heart disease. Atherosclerosis. 1993; 101:1-8.
7. Superko H R, Wood P D, Krauss R M. Effect of alpha- and selective beta-blockade for hypertension control on plasma lipoproteins, apoproteins, lipoprotein subclasses, and postprandial lipemia. Am J Med 1989; 86:26-31.
8. Superko H R. Hypercholesterolemia and dyslipidemia. Current Treatment Options in Cardiovascular Medicine 2000; 2:173-187.
9. Krauss R M, Blanche P J. Detection and quantitation of LDL subfractions. Curr Opin Lipidol 1992:3:377-383.
10. Superko H R. Small, Dense LDL Subclass Pattern B: Issues for Clinicians. Current Atherosclerosis Reports. 1999; 1:50-57.
11. Minihane A M, Khan S, Leigh-Firbank E C, et al. ApoE polymorphism and fish oil supplementation in subjects with an atherogenic lipoprotein phenotype. Arterioscler Thromb Vasc Biol. 2000; 20:1990-1997.
12. Sniderman A D, Cianflone K. Measurement of apoproteins: time to improve the diagnosis and treatment of the atherogenic dyslipoproteinemias. Clin Chem. 1996; 42:489-491
13. O'Keefe J H Jr et al. Effects of pravastatin with NA or magnesium on lipid levels and postprandial lipemia. Am J. Cardiol. 1995 Sep. 1, 76(7):480-484.
14. Brown B G; Zambon A; Poulin D; Rocha A; Maher V M; Davis J W; Albers J J; Brunzell J D. Use of niacin, statins, and resins in patients with combined hyperlipidemia. Am J Cardiol 1998 Feb. 26; 81(4A):52B-59B.
15. Brown G, Albers J J, Fisher L D, Schaefer S M, Lin J T, Kaplan C, Zhao X Q, Bisson B D, Fitzpatrick V F, Dodge H T. Regression of coronary artery disease as a result of intensive lipid-lowering therapy in men with high levels of apolipoprotein B. NEJM 1990; 323:1289-1298.
16. Barter P J, Ballantyne C M, Carmena R, Castro Cabezas M, Chapman M J, Couture P, de Graaf J, Durrington P N, Faergeman O, Frohlich J, Furberg C D, Gagne C, Haffner S M, Humphries S E, Jungner I, Krauss R M, Kwiterovich P, Marcovina S, Packard C J, Pearson T A, Reddy K S, Rosenson R, Sarrafzadegan N, Sniderman A D, Stalenhoef A F, Stein E, Talmud P J, Tonkin A M, Walldius G, Williams K M. Apo B versus cholesterol in estimating cardiovascular risk and in guiding therapy: report of the thirty-person/ten country panel. J Intern Med. 2006 March; 259(3):247-58.
   Guideline 7: HDL Subclass Determination in Patients with CHD or PVD

### Statements Regarding Diagnosis and Management are Suggestions Only and Medical Decision Making Must be Individualized to the Patient.

### Test

HDL subclass distribution

### Background

The HDL subclass test determines HDL subclass distribution and is reported as the amount (mg/dl) and as a percent (% of total HDL) for HDL2 and HDL3. The HDL2 subclass is associated with reduced CHD risk when high and increased CHD risk when low. Low HDL2 levels have been associated with arteriographic severity and progression of CHD. Increasing HDL2 has been associated with improved arteriographic outcomes. Low HDL2 is found more commonly in patients with CHD, premature CHD, a family history of CHD, type 2 diabetes, elevated triglycerides, obesity, and some ethnic groups such as individuals from the subcontinent of India. This may be particularly important in patients with type 2 diabetes mellitus.

The amount and percent distribution has been suggested to reflect the efficiency of reverse cholesterol transport (RCT). Very high or low HDL-C levels are predictive of HDL2 distribution, but a poor correlation exists when HDL-C is approximately 40-55 mg/dl in a man (FIGS. 21, 22 and 24) and 50-65 mg/dl in a woman (FIG. 23).

In patients with CHD, the approximate mean HDL2 distribution is xx mg/dl (xx %) in men and xx mg/dl (xx %) in women. In men with no evidence of CAD the approximate mean value is xx mg/dl (xx %) among those with the small LDL trait, xx mg/dl (xx %) among those with the large LDL trait, and xx mg/dl (xx %) among the middle-aged who exercise regularly. In women who are premenopausal or on postmenopausal hormone replacement therapy (HRT) and have no evidence of CAD, the approximate mean value is xx mg/dl (xx %) among those with the small LDL trait and xx mg/dl (xx %) among those with the large LDL trait.
The basic treatment goal is achieved if HDL-C is >55 mg/dl in men or >65 mg/dl in women who are premenopausal or on HRT. However, in rare instances HDL2 may be low event when HDL-C is above these levels.
Enhance reverse cholesterol transport as reflected by the HDL2 value, in order to assist in the treatment of atherosclerosis.
Consider further increase in HDL2 if evidence of disease progression is apparent.

FIG. 7 illustrates a flowchart for carrying out a seventh guideline in the system and method of the present invention for diagnosing and managing patients at risk for cardiovascular disease.

### Standard HDL-C Measurement.

Be aware that HDL-C measurements can vary considerably in any one patient due to physiologic and laboratory method variability.
   Result #1. HDL-C<40 mg/dl in Men and <50 mg/dl in Premenopausal Women or Women on HRT.
   HDL-C is low and it is likely that the HDL2 will be low as well.

FIG. 21 shows HDL-C and HDL2 in men with no coronary calcification (n=176, r=0.82. p<0.0001, Mean HDLC=45.5+12.9 mg/dl, HDL2b=16.0+7.5%.

FIG. 22 shows HDL-C and HDL2b in men with +CAC (n=173, r=0.74, p<0.0001, Mean HDLC=44.0+12.0, HDL2b=14.7%+6.4%. Note the group of patients with HDLC>55 mg/dl yet low HDL2b (<20%).

FIG. 23 shows HDL-C and HDL2b in women (n=1523, r=0.73, p<0.0001, mean HDLC=

FIG. 24 shows HDL-C and HDL2b in men (n=3, 408, r=0.86, p<0.0001, mean HDLC=

Result #1. HDL-C is Low (<40 mg/dl in M, <50 mg/dl W)
When HDL-C is low it is likely that HDL2 is low.
Consider HDL subclass test to confirm diagnosis of low HDL2.
Consider HDL subclass test as a baseline with which to compare future values.
   Result 1a. Low HDL2 Levels (<xx mg/dl, <xx %)
This supports the disorder of impaired reverse cholesterol transport as suggested by the low HDL-C.
Optimize lifestyle factors that may contribute to low HDL2 and avoid a diet high in simple carbohydrates, avoid nonselective beta blocker medications, control excess body fat, optimize exercise, and optimize blood glucose control (insulin resistance).

Confirm lack of other medical conditions including hypertriglyceridemia, hypothyroidism, renal dysfunction, diabetes, and immunologic disorders.

1) Nicotinic acid (NA) can increase HDL2 by retarding HDL2 degradation and increasing ABCA1 action. NA can be titrated over a wide range to achieve the desired effect.

2) If triglycerides are elevated fibrates can both reduce TG and increase HDL but the effect may be more on HDL3 and may be beneficial.

3) Consider the combination of NA (1,000 to 2,000 mg/d) in combination with a fibric acid derivative (fibrate) which has been reported to achieve a 35% increase in HDL2 (16). Caution regarding drug-drug interactions.

4) Fish oil (EPA) has been reported to increase HDL2.

5) In a high risk patient with low HDL2, consider maintaining a LDL-C in the 50-70 mg/dl range in addition to attempts at increasing HDL2.

6) There may be a different effect of thiozolodinediones, troglitazone has been reported to increase HDL3 while pioglitazone increases HDL2.

7) Avoid Androgenic hormones.

8) Avoid cyclosporine if possible.

9) Avoid selective and nonselective beta blockers if medically appropriate.

10) Consider testing first degree relatives.

Result 1b. “Normal” HDL2 Levels (>xx mg/dl(xx %)<xx mg/dl (xx %))
A “normal” range for risk prediction is not necessarily the optimal level for lipoprotein manipulation intended to improve atherosclerosis.

Consider increasing HDL2 to assist in atherosclerosis treatment.

Consider optimizing exercise, weight management, and reduced carbohydrates in the diet.

If TG is elevated, a reduction in TG is often associated with an increase in HDL2.

1) Nicotinic acid (NA) can increase HDL2 by retarding HDL2 degradation and increasing ABCA1 action. NA can be titrated over a wide range to achieve the desired effect.

2) If TG are elevated, consider a fibric acid derivative.

3) Fish oil (EPA) has been reported to increase HDL2.

4) In a high risk patient with low HDL2, consider maintaining a LDL-C in the 50-70 mg/dl range in addition to attempts at increasing HDL2.

5) Avoid Androgenic hormones.

6) Test first degree relatives for low HDL-C.

Result 1c. Elevated 1DL2 Levels>xx mg/dl, xx %.
It is unlikely that disorders of HDL subclass distribution are contributing to atherosclerosis risk.
Consider optimizing exercise, weight management and reduced carbohydrates in the diet to increase HDL-C.
Consider NA to increase HDL-C or NA in combination with a fibrate.
Avoid androgens.
Test for other metabolic disorders contributing to atherosclerosis.

### Result #2. HDL-C is in What is Considered to be a “Normal” Range (≧40<65 M, ≧50<75 W)

Within this range it is very difficult to predict the probability of low or high HDL2.
Patients within this HDL-C range may benefit from HDL subclass testing if the results are utilized to:
Make a diagnosis.
Help determine risk prediction.
Use as a baseline.
Help select lipid therapy.
Consider the effect of beta blocker on HDL subclass distribution.
   Result 2a. Low HDL2 Levels (<xx mg/dl, <xx %)
This suggests the disorder of impaired reverse cholesterol transport.
Optimize lifestyle factors that may contribute to low HDL2 and avoid a diet high in simple carbohydrates, avoid nonselective beta blocker medications, control excess body fat and optimize exercise, and optimize blood glucose control (insulin resistance).

Confirm lack of other medical conditions including hypertriglyceridemia, hypothyroidism, renal dysfunction, diabetes, and immunologic disorders.

1) Nicotinic acid (NA) can increase HDL2 by retarding HDL2 degradation and increasing ABCA1 action. NA can be titrated over a wide range to achieve the desired effect.

2) If triglycerides are elevated fibrates can both reduce TG and increase HDL but the effect may be more on HDL3 and may be beneficial.

3) Consider the combination of NA (1,000 to 2,000 mg/d) in combination with a fibric acid derivative (fibrate) which has been reported to achieve a xx % increase in HDL2. Caution regarding drug-drug interactions.

4) Fish oil (EPA) has been reported to increase HDL2.

5) In a high risk patient with low HDL2, consider maintaining a LDL-C in the 50-70 mg/dl range in addition to attempts at increasing HDL2.

6) There may be a different effect of thiozolodinediones, troglitazone has been reported to increase HDL3 while pioglitazone increases HDL2.

7) Avoid Androgenic hormones.

8) Avoid cyclosporine if possible.

9) Avoid selective and nonselective beta blockers if medically appropriate.

10) Consider testing first degree relatives.

Result 2b. “Normal” HDL2 Levels (>xx mg/dl(xx %)<xx mg/dl (xx %))
A “normal” range for risk prediction is not necessarily the optimal level for lipoprotein manipulation intended to improve atherosclerosis.

Consider increasing HDL2 to assist in atherosclerosis treatment.

Consider optimizing exercise, weight management, and reduced carbohydrates in the diet.

If TG is elevated, a reduction in TG is often associated with an increase in HDL2.

Consider:

1) Nicotinic acid (NA) can increase HDL2 by retarding HDL2 degradation and increasing ABCA1 action. NA can be titrated over a wide range to achieve the desired effect.

2) Fish oil (EPA) has been reported to increase HDL2.

3) Avoid Androgenic hormones.

Result 2c. Elevated HDL2 Levels>xx mg/dl, xx %.
It is unlikely that impaired reverse cholesterol transport is contributing to atherosclerosis risk.

Test for other metabolic disorders contributing to atherosclerosis.

Result #3. Elevated HDL-C (>65 mg/dl in Males, >75 mg/dl in females)
Reflects no major impairment in RCT, however, optimization of RCT may provide some protection from CHD progression.
It is unlikely that HDL2 is low.
Consider HDL subclass test is clinical suspicion exists such as premature CHD or ethnicity reported to have low HDL2 in the presence of normal HDL-C.
Investigate other metabolic causes of atherosclerosis.
Low dose NA (1,000 mg/d) may be effective in retarding progression of atherosclerosis even more than statin alone.
   Result 3a. Low HDL2 Levels (<xx mg/dl, <xx %)
The implication of low HDL2 in the setting of elevated HDL-C is unclear.
Search for and treat other disorders associated with CHD.
If no other disorders are present, attempts to increase HDL2 may be useful but clinical trial data is sparse.
Optimize lifestyle factors that may contribute to low HDL2 and avoid a diet high in simple carbohydrates, avoid nonselective beta blocker medications, control excess body fat and optimize exercise, and optimize blood glucose control (insulin resistance).

1. Nicotinic acid (NA) can increase HDL2 by retarding HDL2 degradation and increasing ABCA1 action. NA can be titrated over a wide range to achieve the desired effect.

2. Avoid Androgenic hormones.

3. Avoid selective and nonselective beta blockers if medically appropriate.

Result 3b. “Normal” HDL2 Levels (>xx mg/dl(xx %)<xx mg/dl (xx %))
A “normal” HDL2 in the setting of elevated HDL-C suggests other disorders are present that contribute to CHD.
Investigate other metabolic causes of atherosclerosis.

1) Nicotinic acid (NA) can increase HDL2 by retarding HDL2 degradation and increasing ABCA1 action. NA can be titrated over a wide range to achieve the desired effect.

2) Fish oil (EPA) has been reported to increase HDL2.

3) Avoid Androgenic hormones.

Result 3c. Elevated HDL2 Levels>xx mg/dl, xx %.
It is unlikely that impaired reverse cholesterol transport is contributing to atherosclerosis risk.

1) Test for other metabolic disorders contributing to atherosclerosis.

### INCORPORATED REFERENCES

1. Superko H R. Lipoprotein Subclasses and Atherosclerosis. Frontiers in Bioscience 2001; 6:d355-365 (Mar. 1, 2001).
2. Campos H, Roederer G O, Lussier-Cacan S, Davignon J, Krauss R M. Predominance of large LDL and reduced HDL2 cholesterol in normolipidmeic men with CAD. Arterioscler Thromb Vase Biol 1995; 15:1043-1048.
3. Miller N E. Associations of high-density lipoprotein subclasses and apolipoproteins with ischemic heart disease and coronary atherosclerosis. Am Heart J 1987; 113:589-597.
4. Franceschini G, Bondioli A, Granata D, Mercuri V, Negri M, Tosi C, Sirtori C R. Reduced HDL2 levels in myocardial infarction patients without risk factors for atherosclerosis. Atherosclerosis 1987; 68:213-219
5. Johansson J, Carlson L A, Landow C, Hamsten A. High density lipoproteins and coronary atherosclerosis. A strong inverse relation with the largest particles is confined to normotriglyceridemic patients. Arterioscler Thromb 1991; 11:174-182.
6. Brown G B, Zhao X Q, Chait A, Fisher L D, Cheung M C, Morse J S, Dowdy A A, Marino E K, Bolson E L, Alaupovic P, Frohlich J, Albers J J. Simvastatin and niacin, antioxidant vitamins, or the combination for the prevention of coronary disease. NEJM 2001; 345:1583-1592.
7. Superko H R. The Failure of LDL Cholesterol Reduction and the Importance of Reverse Cholesterol Transport. The Role of Nicotinic Acid. Br J Card 2006; 13:131-136.
8. Laakso M, Voutilainen E, Pyorala K, Sarlund H. Association of low HDL and HDL2 cholesterol with coronary heart disease in noninsulin-dependent diabetoics. Arteriosclerosis 1985; 5:653-658.
9. Dreon D M, Fernstrom H, Miller B, Krauss R M. Low density lipoprotein subclass patterns and lipoprotein response to a reduced-fat diet in men. FASEB J. 1994; 8:121-126.
10. Superko H R, Wood P D, Krauss R M. Effect of alpha- and selective beta-blockade for hypertension control on plasma lipoproteins, apoproteins, lipoprotein subclasses, and postprandial lipemia. Am J Med 1989; 86:26-31.
11. H. Robert Superko, M D, Ronald M. Krauss, M D, William L. Haskell, PhD, Stanford Coronary Risk Intervention Project Investigators. Association of lipoprotein subclass distribution with use of selective and non-selective beta-blocker medications in patients with coronary heart disease. Atherosclerosis 1993; 101: 1-8.
12. Williams P T, Krauss R M, Vranizan K M, Wood P D. Changes in lipoprotein subfractions during diet-induced and exercise-induced weight loss in moderately overweight men. Circulation 1990; 81:1293-1304.
13. Laakso M, Voutilainen E, Pyorala K, Sarlund H. Association of low HDL and HDL2 cholesterol with coronary heart disease in noninsulin-dependent diabetoics. Arteriosclerosis 1985; 5:653-658.
14. Meyers C D, Kashyap M L. Pharmacologic augmentation of high-density lipoproteins: mechanisms of currently available and emerging therapies. Curr Opin Cardiol.
2005 July; 20(4):307-12.
15. Carlson L A. Nicotinic acid: the broad-spectrum lipid drug. A 50th anniversary review. J Intern Med. 2005 August; 258(2):94-114.
16. Superko H R, Berneis, K K, Williams, P T, Rizzo, M, Wood, P D. Differential Effect of Gemfibrozil in Normolipemic Subjects with Predominantly Dense or Buoyant Low Density Lipoprotein Particles and the Effect on Postprandial Lipemia and Lp(a). Am J Cardiology 2005; 96:1266-1272.
17. Petersen M, Pedersen H, Major-Pedersen A, Jensen T, Marckmann P. Effect of fish oil versus corn oil supplementation on LDL and HDL subclasses in type 2 diabetic patients. Diabetes Care. 2002 October; 25(10):1704-8.
18. Gomez-Perez F J, Aguilar-Salinas Calif., Vazquez-Chavez C, Fanghanel-Salmon G, Gallegos-Martinez J, Gomez-Diaz R A, Salinas-Orozco S, Chavira-Lopez I J, Sanchez-Reyes L, Torres-Acosta E M, Tamez R, Lopez A, Guillen L E, Cesarman G. Further insight on the hypoglycemic and nonhypoglycemic effects of troglitazone 400 or 600 mg/d: effects on the very-low-density and high-density lipoprotein particle distribution. Metabolism. 2002 January; 51(1):44-51.
19. Lawrence J M, Reid J, Taylor G J, Stirling C, Reckless J P. Favorable effects of pioglitazone and metformin compared with gliclazide on lipoprotein subfractions in overweight patients with early type 2 diabetes. Diabetes Care. 2004 January; 27(1):41-6.
20. Kantor M A, Bianchini A, Bernier D, Sady S P, Thompson P D. Androgens reduce HDL2-cholesterol and increase hepatic triglyceride lipase activity. Med and Sci in Sports and Ex 1985; 17:462-465.
21. H. Robert Superko, William L. Haskell, Connie D. Di Ricco. Lipoprotein and hepatic lipase activity and high density lipoprotein subclasses after cardiac transplantation. Am J Card. 1990; 66:1131-1134.
22. Williams P T, Vranizan K M, Austin M A, Krauss R M. Familial correlations of HDL subclasses based on gradient gel electrophoresis. Arterioscerosis and Thrombosis 1992; 12:1467-1474.
23. Bell D S. A comparison of lovastatin, an HMG-CoA reductase inhibitor, with gemfibrozil, a fibrinic acid derivative, in the treatment of patients with diabetic dyslipidemia. Clin Ther. 1995 September-October; 17(5):901-10.
24. Syvanne M, Nieminen M S, Frick M H, Kauma H, Majahalme S, Virtanen V, Kesaniemi Y A, Pastemack A, Ehnholm C, Taskinen M R. Associations between lipoproteins and the progression of coronary and vein-graft atherosclerosis in a controlled trial with gemfibrozil in men with low baseline levels of HDL cholesterol. Circulation. 1998 Nov. 10; 98(19):1993-9.
25. Taylor A J, Sullenberger L E, Lee H J, Lee J K, Grace K A. Arterial Biology for the Investigation of the Treatment Effects of Reducing Cholesterol (ARBITER) 2: a double-blind, placebo-controlled study of extended-release niacin on atherosclerosis progression in secondary prevention patients treated with statins. Circulation. 2004 Dec. 7; 110(23):3512-7.
26. Superko H R, Bachorik P S, Wood P D. High-density lipoprotein cholesterol measurements. JAMA 256:2714-2717, 1986.
27. Superko H R, Enas E A, Kotha P, Bhat N K, Garrett B. HDL subclass distribution in individuals of Asian Indian descent. The National Asian Indian Heart Disease Project. Preventive Cardiology 2004:8:81-86.
   Guideline 8: Fasting Triglycerides 400 mg/dl-1,000 mg/dl

### Statements Regarding Diagnosis and Management are Suggestions Only and Medical Decision Making Must be Individualized to the Patient.

### Test:

Fasting Triglycerides

### Background

Fasting triglyceride values>400 mg/dl and <1,000 mg/dl often are the result of polygenic environment interactions. CHD risk can vary considerably depending on the underlying cause of the hypertriglyceridemia. Normal lipoprotein lipase (LPL) function is essential for normal triglyceride hydrolysis, and apolipoprotein C-II is a cofactor for LPL action. Over 26 mutations in the LPL gene have been identified that can result in a spectrum between mild to complete LPL activity deficiency (3). Apolipoprotein C-II is a cofactor for LPL action. Substantial elevation in chylomicrons and VLDL are seen in patients lacking this apoprotein (4). The heterozygote condition is present in approximately 12% of Americans of African ancestry (8).

Elevated triglycerides can be exacerbated by several factors including thyroid function, renal function, diabetes, use of beta blockers and some steroids, diets rich in simple carbohydrates, overweight status, and alcohol consumption.
Rule out thyroid dysfunction, renal disease, diabetes, excess calorie consumption, overweight status, and any alcohol consumption.
Avoid simple carbohydrates in the diet and consider a Mediterranean diet.
Reduce excess body fat.
Reduce dietary calories.
Optimize physical activity.
Consider a fish oil supplement.
Consider Nicotinic Acid (NA).
Consider a fibrate if NA is not tolerated or contraindicated.
Consider screening first degree family members.

### Repeat Fasting Triglycerides

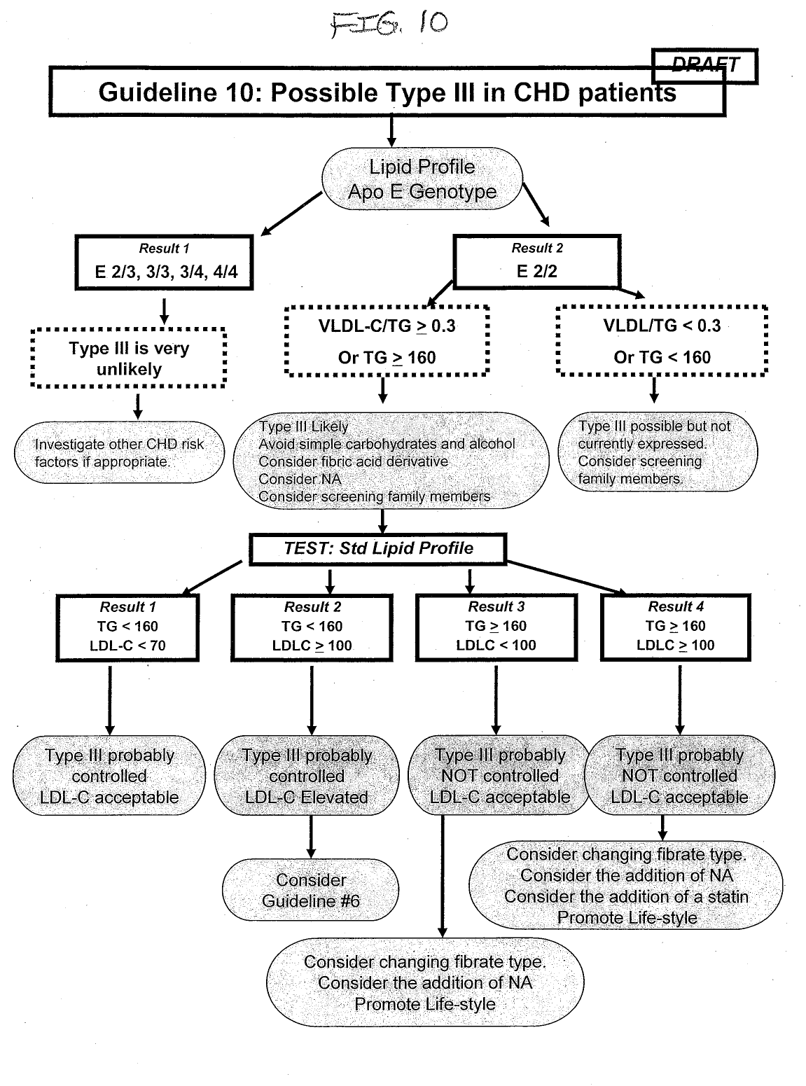

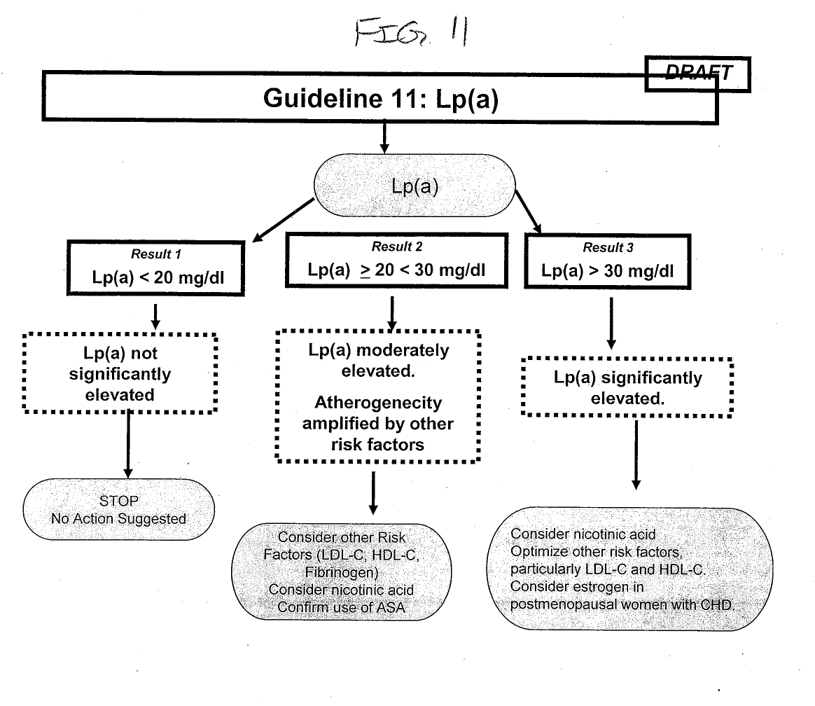

FIG. 8 illustrates a flowchart for carrying out an eighth guideline in the system and method of the present invention for diagnosing and managing patients at risk for cardiovascular disease.

Result #1. TG<400, LDLC<100 mg/dl.

Consider guideline # x.

Repeat LDL subclass test
If LDL pattern A:
Consider increasing the statin dose or switching to a more powerful statin
Consider ezetimibe+statin to assist LDL-C reduction.
Consider a low-dose bile-acid-binding resin to assist LDL-C reduction.
If LDL pattern B:
Optimize lifestyle.
Consider titrating NA dose to a higher level.
Consider adding a fibrate if medically acceptable.
Consider increasing the statin dose or switching to a more powerful statin.
Consider ezetimibe+statin to assist LDL-C reduction.
Consider a low-dose bile-acid-binding resin to assist LDL-C reduction.
Consider adding a fish oil supplement.
Monitor for potential drug interactions.
   Result #2. LDL-C<70 mg/dl, TG≧100 mg/dl.

Repeat LDL subclass test
If LDL pattern A, the goal is achieved.
If LDL pattern B:
Optimize lifestyle.
Consider titrating NA dose to a higher level.
Consider adding a fibrate if medically acceptable.
Consider adding a fish oil supplement.
Monitor for potential drug interactions.
   Result #3. LDL-C>70 mg/dl and TG<100 mg/dl.
Consider increasing the statin dose or switching to a more powerful statin.
Consider ezetimibe+statin to assist LDL-C reduction.
Consider a low-dose bile-acid-binding resin to assist LDL-C reduction.

### INCORPORATED REFERENCES

1. Santamarina-Fojo S, Brewer H B Jr. The familial hyperchylomicronemia syndrome. New Insights into underlying genetic defects. JAMA 1991; 265:904-908.
2. Gagne C, Brum L D, Julien P, Moorjani S, Lupien P J. Primary lipoprotein lipase activity deficiency. Clinical investigation of a French Canadian population. Can Med Assoc J 1989; 140:405-411.
3. Santamarina-Fojo S. Genetic dyslipoproetinemias: role of lipoprotein lipase and apolipoprotein C-II. Current Opinion in Lipidology 1992; 3:186-195.
4. Santamarina-Fojo S. Genetic dyslipoproetinemias: role of lipoprotein lipase and apolipoprotein C-II. Current Opinion in Lipidology 1992; 3:186-195.
5. Breckenridge W C, Alaupovic P, Cox D W, et al: Apoprotein and lipoprotein concentrations in familial apolipoprotein C-II deficiency. Atherosclerosis 44:223-235, 1982.
6. Windier E, Preyer S, Greten H: Changes in affinity of triglyceride-rich lipoproteins to apolipoprotein C-II during lipolysis. In Carlson L A Olsson A G (eds): Treatment of Hyperlipoproteinemia. New York, Raven Press, 1984, 00 95-98.
7. Miller N E, Rao S M, Alaupovic P, et al: Familial apolipoprotein C-II deficiency; plasma lipoproteins in heterozygous and homozygous subjects and the effects of plasma infusion. Eur J Clin Invest 11:69-76, 1981.
8. Menzel H J, Kane J P, Malloy M J, Havel R J. A varian primary structure of apolipoprotein C-II in individuals of African Descent. J Clin Invest 1986; 77:595-601.
9. Brown B G; Zambon A; Poulin D; Rocha A; Maher V M; Davis J W; Albers J J; Brunzell J D. Use of niacin, statins, and resins in patients with combined hyperlipidemia. Am J Cardiol 1998 Feb. 26; 81(4A):52B-59B.
10. Brown G, Albers J J, Fisher L D, Schaefer S M, Lin J T, Kaplan C, Zhao X Q, Bisson B D, Fitzpatrick V F, Dodge H T. Regression of coronary artery disease as a result of intensive lipid-lowering therapy in men with high levels of apolipoprotein B. NEJM 1990; 323: 1289-1298.
   Guideline #9: Triglycerides>1,000 mg/di

### Statements Regarding Diagnosis and Management are Suggestions Only and Medical Decision Making Must be Individualized to the Patient.

### Test

### Fasting Triglycerides

### Background

Fasting triglyceride values>1,000 mg/dl greatly increase the risk for pancreatitis. At this level, laboratory accuracy is problematic and triglyceride changes of 100 mg/dl probably do not reflect significant physiologic or real change. Elevated triglycerides can be exacerbated by several factors including thyroid function, renal function, diabetes, use of beta blockers and some steroids, diets rich in simple carbohydrates as well as diets rich in fats, overweight status, and alcohol consumption.

While mild to moderate elevations in plasma triglycerides is often a polygenic environment interaction, dramatic elevations in fasting triglycerides, usually greater than 1,000 mg/dl, are often associated with inherited defects in triglyceride metabolism. Normal lipoprotein lipase (LPL) function is essential for normal triglyceride hydrolysis, and apolipoprotein C-II is a cofactor for LPL action. LPL deficiency is the most common cause of familial chylomcronemia and is an autosomal recessive trait which often presents in childhood with severely elevated plasma triglycerides, pancreatitis and abdominal pain, eruptive xanthomas and lipemia retinales. It occurs in approximately 1:5,000 individuals and the heterozygote state is more common than the classic inherited cholesterol disorder, familial heterozygote hypercholesterolemia, and in some populations can be found in 1 in 40 individuals. Over 26 mutations in the LPL gene have been identified that can result in a spectrum between mild to complete LPL activity deficiency. Apolipoprotein C-II is a cofactor for LPL action. Substantial elevation in chylomicrons and VLDL is seen in patients lacking this apoprotein. Homozygote patients lacking apoprotein C-II have markedly decreased LDL and HDL values, which supports its role as a cofactor in the conversion of chylomicrons and VLDL to denser lipoproteins. Apo C-II deficiency is a rare disorder inherited in an autosomal recessive manner and presents clinically in a similar manner as LPL deficiency. However, unlike LPL deficiency, infusion of normal plasma will temporarily reduce plasma triglycerides. The heterozygote condition is present in approximately 12% of Americans of African ancestry.
Consider an endocrinology consult to help rule out primary causes of severe hypertriglyceridemia.
Rule out thyroid dysfunction, renal disease, diabetes, excess calorie consumption, and any alcohol consumption.
Reduce triglycerides to <1,000 mg/dl rapidly in order to help prevent pancreatitis.
Avoid simple carbohydrates in the diet and consider a Mediteranean diet.
Reduce excess body fat.
Reduce dietary calories.
Optimize physical activity.
Consider a fish oil supplement.
Consider Nicotinic Acid (NA).
Consider a fibrate if NA is not tolerated or contraindicated.
Consider NA+statin.
Consider screening first degree family members.

### Repeat Fasting Triglycerides

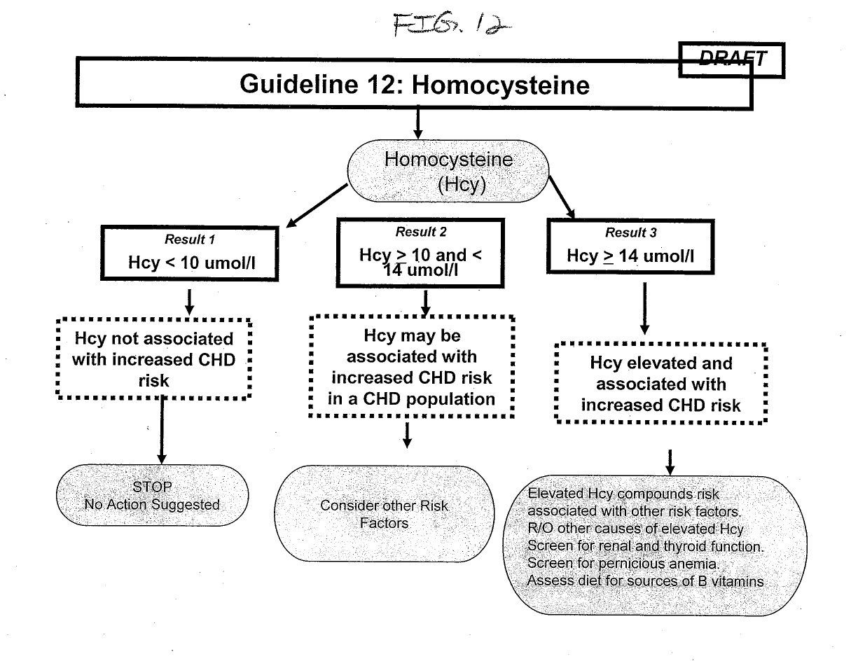

FIG. 9 illustrates a flowchart for carrying out a ninth guideline in the system and method of the present invention for diagnosing and managing patients at risk for cardiovascular disease.

Result #1. TG<100, LDLC<70 mg/dl.

Goal achieved.

Monitor standard lipid profile and safety tests.

Result #2. TG>100 mg/dl, LDL-C<70 mg/dl.

Repeat LDL subclass test
If LDL pattern A, the goal is achieved.
If LDL pattern B:
   Optimize lifestyle.
   Consider titrating NA dose to a higher level.
   Consider adding a fibrate if medically acceptable.
   Consider adding a fish oil supplement.
   Monitor for potential drug interactions.
      Result #3. TG<100 mg/dl and LDL-C≧70 mg/dl.
Consider increasing the statin dose or switching to a more powerful statin.
Consider ezetimibe+statin to assist LDL-C reduction.
Consider a low-dose bile-acid-binding resin to assist LDL-C reduction.
   Result #4. TG>100 mg/dl and >70 mg/dl.
Consider Guideline X

### RELATED READING INCORPORATED BY REFERENCE

1. Santamarina-Fojo S, Brewer H B Jr. The familial hyperchylomicronemia syndrome. New Insights into underlying genetic defects. JAMA 1991; 265:904-908.
2. Gagne C, Brum L D, Julien P, Moorjani S, Lupien P J. Primary lipoprotein lipase activity deficiency. Clinical investigation of a French Canadian population. Can Med Assoc J 1989; 140:405-411.
3. Santamarina-Fojo S. Genetic dyslipoproetinemias: role of lipoprotein lipase and apolipoprotein C-II. Current Opinion in Lipidology 1992; 3:186-195.
4. Santamarina-Fojo S. Genetic dyslipoproetinemias: role of lipoprotein lipase and apolipoprotein C-II. Current Opinion in Lipidology 1992; 3:186-195.
5. Breckenridge W C, Alaupovic P, Cox D W, et al: Apoprotein and lipoprotein concentrations in familial apolipoprotein C-II deficiency. Atherosclerosis 44:223-235, 1982.
6. Windier E, Preyer S, Greten H: Changes in affinity of triglyceride-rich lipoproteins to apolipoprotein C-II during lipolysis. In Carlson L A Olsson A G (eds): Treatment of Hyperlipoproteinemia. New York, Raven Press, 1984, 00 95-98.
7. Miller N E, Rao S M, Alaupovic P, et al: Familial apolipoprotein C-II deficiency; plasma lipoproteins in heterozygous and homozygous subjects and the effects of plasma infusion. Eur J Clin Invest 11:69-76, 1981.
8. Menzel H J, Kane J P, Malloy M J, Havel R J. A varian primary structure of apolipoprotein C-II in individuals of African Descent. J Clin Invest 1986; 77:595-601.
9. Brown B G; Zambon A; Poulin D; Rocha A; Maher V M; Davis J W; Albers J J; Brunzell J D. Use of niacin, statins, and resins in patients with combined hyperlipidemia. Am J Cardiol 1998 Feb. 26; 81(4A):52B-59B.
10. Brown G, Albers J J, Fisher L D, Schaefer S M, Lin J T, Kaplan C, Zhao X Q, Bisson B D, Fitzpatrick V F, Dodge H T. Regression of coronary artery disease as a result of intensive lipid-lowering therapy in men with high levels of apolipoprotein B. NEJM 1990; 323:1289-1298.
   Guideline #10. CAD Patient with Possible Type III Hyperlipidemia: Apo E2/E2+TG≧A60 mg/dl

### Statements Regarding Diagnosis and Management are Suggestions Only and Medical Decision Making Must be Individualized to the Patient.

### Test

Fasting Triglycerides
Apo E genotype

### Background

The disorder, Type III Hyperlipoproteinemia, is an example of an interaction of the apoE2 homozygous state with another genetic or environmental factor leading to marked accumulation of triglyceride-rich lipoprotein remnants and accelerated atherosclerosis. Approximately 1:5,000 individuals may express this disorder. Over 90% of individuals with Type III hyperlipoproteinemia are apoE2 homozygotes; however, the disease is caused by interaction of the apoE2/E2 state with another genetic or environmental factor because while about 1% of the population expresses the E2/2 isoform, only 2% of these develop Type III Hyperlipidemia and most individuals with E2/E2 do not exhibit the abnormal lipid profile. Expression is suggested by the presence of a triglyceride (TG) level≧160 mg/dl in combination with the apo E2/E2 genotype.

Two established alternative indicators of type III hyperlipidemia are:
A ratio of VLDL-C (directly measured by ultracentrifugation rather than calculated as TG/5) to fasting TG that exceeds 0.3.
Demonstration of a “floating beta,” on agarose gel electrophoresis (an old laboratory method).

Individuals with type III HLP have increased risk for CHD events.

Tuberous and tendinous xanthomata can be found in patients with Type III HLP.

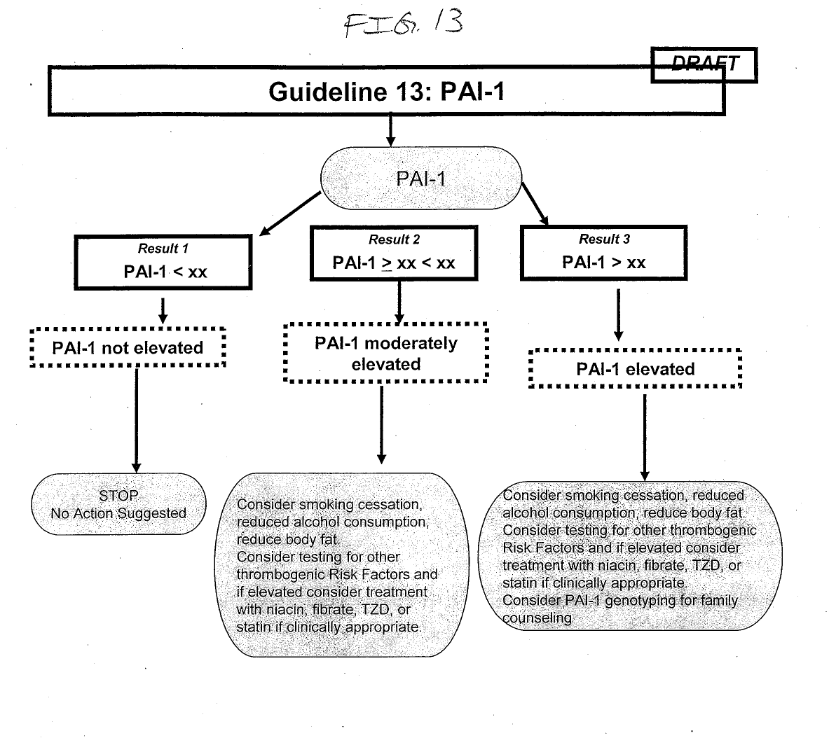

FIG. 10 illustrates a flowchart for carrying out a tenth guideline in the system and method of the present invention for diagnosing and managing patients at risk for cardiovascular disease.

### Result #1. Apo E Genotype 2/3, 3/3, 3/4, 4/4.

It is very unlikely that Type III HLP can exist since the Apo E 2/2 genotype does not appear to be present.

### Result #2. Apo E Genotype 2/2.

Type III HLP is possible.
If VLDL-C/TG is >0.3 or TG>160 mg/dl, a presumptive diagnosis of type III HLP can be made.
The presence of a “floating beta” on agarose gel electrophoresis was the traditional diagnostic criteria in the past.
This condition can be greatly exacerbated by excess calories in the diet, including those derived from alcohol. It is important to reduce excess body fat and optimize daily physical activity.
Consider gemfibrozil or fenofibrate as medications. Clofibrate has been successfully used in type III hyperlipidemia. Be aware that fibric acid treatment can cause a rise in LDL-C as TG is reduced.
Consider a fibrate and nicotinic acid combination.
Consider screening first degree relatives with fasting TG and apo E genotype if appropriate.

### Repeat Laboratory Testing

Result #1. TG<160 mg/dl, LDL-C<100 mg/dl.

Goal for Type III expression achieved.

Consider the presence of other disorders contributing to CHD risk.

Result #2. TG<160 mg/dl, LDL-C>100 mg/dl.

Refer to Guideline #6.

Result #3. TG>160 mg/dl, LDL-C<100 mg/dl.
The elevated TG suggests that type III hyperlipidemia continues to be expressed.
Consider substituting clofibrate for gemfibrozil or fenofibrate, balancing the potential for reduction in cardiovascular events against the reported association of clofibrate with adverse side effects.
If clofibrate is not acceptable, consider adding nicotinic acid (NA) to gemfibrozil or fenofibrate.
Continue to promote life-style treatment.
Consider fish oil supplements.
Avoid all alcohol consumption.
   Result #4. TG>160 mg/dl, LDL-C≧100 mg/dl.
The elevated TG suggests that type III hyperlipidemia continues to be expressed.
Consider substituting clofibrate for gemfibrozil or fenofibrate, balancing the potential for reduction in cardiovascular events against the reported association of clofibrate with adverse side effects.
If clofibrate is not acceptable, consider adding nicotinic acid (NA) to gemfibrozil or fenofibrate.
Consider the addition of a statin with appropriate caution to potential drug interactions.
Continue to promote life-style treatment.
Consider fish oil supplements.
Avoid all alcohol consumption.

### RELATED READING INCORPORATED BY REFERENCE

1. Mahley R W, Innerarity T L, Rall S C, et al. Apolipoprotein E: genetic variants provide insights into its structure and function. Curr Opin Lipidol. 1990; 1:87-95.
2. Patsch J R, Jackson R L, Gotto A M Jr. Evaluation of the classical methods for the diagnosis of type III hyperlipoproteinemia. Klin Wochenschr. 1977; 55:1025-1030.
3. American College of Medical Genetics/American Society of Human Genetics Working Group on ApoE and Alzheimer Disease. Statement on use of apoliporptein E testing for alzheimer disease. JAMA 1995; 274:1627-1629.
4. Minihane A M, Khan S, Leigh-Firbank E C, Talmud P, Wright J W, Murphy M C, Griffin B A, Williams C M. ApoE polymorphism and fish oil supplementation in subjects with an atherogenic lipoprotein phenotype. Arterioscler Thromb Vasc Biol 2000; 20:1990-1997.
5. Cumming A M, Robertson F. Polymorphism at the apo E locus in relation to risk or coronary disease. Clin Genet 25:310-313, 1984.

### Guideline #11. Lipoprotein (a) Level in CHD Patients

### Statements Regarding Diagnosis and Management are Suggestions Only and Medical Decision Making Must be Individualized to the Patient.

### Test

Lp(a)

### Background

Lipoprotein little (a) (Lp(a)) is an LDL particle with the protein apo (a) attached by a disulfide bridge. [a] is a protein with structural similarities to plasminogen. Elevated Lp(a) has a strong association with CHD and atherosclerosis in the carotid arteries, and may be elevated in as many as 20-40% of individuals with CHD. Rapid progression of arteriographically quantitated CHD has been reported to be significantly more common in subjects with elevated Lp(a), is an independent risk factor for myocardial infarction in young men, is independently associated with arteriographically defined coronary disease, and has been reported to be more closely linked to the extent of coronary atherosclerosis than other lipid parameters. CHD risk associated with Lp(a) is amplified by other risk factors such as elevated LDL-C or low HDL-C. It is transmitted in a dominant fashion and approximately 50% of first degree relative of patients with elevated Lp(a) will also express this disorder.

While the evidence that Lp(a) has a strong link to CHD and PVD is abundant, the clinical evidence that reducing Lp(a) is beneficial to the patient is sparse. Reduction of elevated Lp(a) following apheresis has been associated with a significant reduction in restenosis following PTCA. Reduction of elevated Lp(a) in post menopausal women with HRT has been associated with a statistically significant reduction in CV events. Retrospective evidence from the FATS trial suggests that significant reduction of LDL-C in patients with elevated Lp(a) may retard the rate of arteriographic progression even though Lp(a) levels are not significantly reduced.

The risk of elevated Lp(a) associated with a specific blood level is related to different Lp(a) isoforms and genotypes. The (a) protein can vary in size and in general, the smaller the (a), the greater the CHD risk and impaired blood vessel flow mediated dilation. An ethnic difference has been described in which Afro-American men have larger (a) size compared to Caucasian men and this may help explain the relatively lower CHD risk associated with a given Lp(a) levels in AfroAmerican males compared to Caucasian males. Similar differences have also been noted in different Native Mexican population.

In general, nicotinic acid (NA), or estrogen in a postmenopausal woman, can reduce elevated Lp(a). NA doses of 2000 to 5000 mg/d are often required for efficacy and may reduce Lp(a) approximately 50%. Estrogen can reduce Lp(a) approximately 20-30%. A Lp(a) of 20 mg/dl is approximately the 90th percentile in a healthy population, and the threshold for increased CHD risk is generally observed between 20-30 mg/dl. Laboratory methods vary and may result in different values considered to be at risk for a cardiovascular event.

### Initial Action

Start NA 1500 mg/d or postmenopausal estrogen treatment if clinically appropriate.
Assess and optimally treat other risk factors.

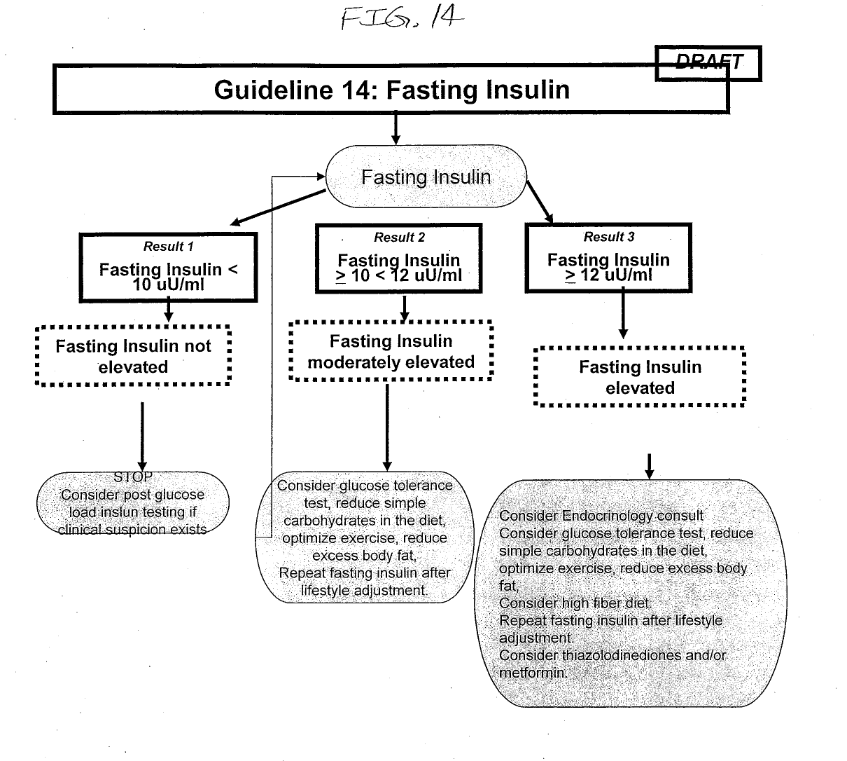

FIG. 11 illustrates a flowchart for carrying out an eleventh guideline in the system and method of the present invention for diagnosing and managing patients at risk for cardiovascular disease.

Result #1. Lp(a)<20 mg/dl.
Lp(a) is probably <the 90th percentile and within an acceptable range. However, the final goal for each patient should be individualized based on the medical history and other risk factors.
   Result #2. Lp(a)≧20 mg/dl.

Consider further but tolerable increases in NA to reduce Lp(a).
If the Lp(a) cannot be reduced, consider optimizing other risk factors. Using a statin, ±ezetimibe, ±bile-acid-binding resin to reduce LDL-C to 50-70 mg/dl may be beneficial.
Neomycin therapy has been shown to reduce Lp(a), but caution is advised because of this drug's reported association with auditory loss.
Because oxidation of Lp(a) increases macrophage uptake of the Lp(a) particle, antioxidant treatment with vitamins E and C may provide some degree of protection. The benefit of this treatment in humans has not been demonstrated and may adversely impact the ability of NA to increase HDL2 and reduce CHD events and progression.

### RELATED READING INCORPORATED BY REFERENCE

1. Scanu A M, Gless G M. Lipoprotein (a). Heterogeneity and biological relevance. J Clin Invest 1990; 85:1709-1715.
2. Marquez A, Mendoza S, Hamer T, et al. High Lp(a) in children from kindreds with parental premature myocardial infarction. Circulation 1993; 88:1-97 (abstr).
3. Terres W, Tatsis E, Pfalzer B, Beil U, Beisiegel U, Hamm C W. Rapid angiographic progression of coronary artery disease in patients with elevated lipoprotein (a). Circulation 1995; 91:948-950.
4. Sandkamp M, Funke H, Schelte H, Kohler E, Assmann G. Lipoprotein(a) is an independent risk factor for myocardial infarction at a young age. Clin Chem 1990; 36:20-23.
5. Dahlen G H, Guyton J R, Attar M, et al. Association of levels of lipoprotein Lp(a), plasma lipids, and other lipoproteins with coronary artery disease documented by angiography. Circulation 1986; 74:758-765.
6. Budde T, Fechtrup C, Bosenberg E, et al. Plasma lp(a) levels correlate with number, severity, and length-extension of coronary lesions in male patients undergoing coronary arteriography for clinically suspected coronary atherosclerosis. Arterioscler Thromb 1994; 14:1730-1736.
7. Schaefer E J, Lamon-Fava S, Jenner J L, McNamara J R, Ordovas J M, Davis E, Abolafia J M, Lippel K, Levy R I. Lipoprotein(a) levels and risk of coronary heart disease in men. JAMA 1994; 271:999-1003.
8. Naruszewicze M, Giroux, L M, Davignon J. Oxidative modification of Lp(a) causes changes in the structure and biological properties of apo (a). Chem Phys Lipids 1994; 67-68; 167-174.
9. Daida H, Lee Y J, Yokoi H, Kanoh T, Ishiwata S, Kato K, Nishikawa H, Takatsu F, Kato H, Kutsumi Y, Yamada N, Noma A, Yamaguchi H. Prevention of restenos after percutaneous transluminal coronary angioplasty by reducing lipoprotein (a) levels with low-density lipoprotein apheresis. Am J Card 1994; 73:1037-1040.
10. Shlipak M G, Simon J A, Vittinghoff E, Lin F, Barrett-Conner E, Knopp R H, Levy, R I, Hulley S B. Estrogen and Progestin, Lipoprotein(a), and the Risk of Recurrent Coronary Heart Disease Events After Menopause. JAMA 2000; 283:1845-1852.
11. Brewer H B Jr. Effectiveness of diet and drugs in the treatment of patients with elevated Lp(a) levels. In Scanu A M ed. Lipoprotein (a). New York, N.Y.: Academic Press 1990:211-220.
12. Gurakar A, Hoeg J M, Kostner G, Papadopoulos N M, Brewer H B Jr. Levels of lipoprotein Lp(a) decline with neomycin and niacin treatment. Atherosclerosis. 1985:57; 293-301.
13. Brown G B, Zhao X Q, Chait A, Fisher L D, Cheung M C, Morse J S, Dowdy A A, Marino E K, Bolson E L, Alaupovic P, Frohlich J, Albers J J. Simvastatin and niacin, antioxidant vitamins, or the combination for the prevention of coronary disease. NEJM 2001; 345:1583-1592.
14. Cantin B, Gagnon F, Moorjani S, Despres J P, Lamarche B, Lupien P J, Dagenais G R. Is lipoprotein(a) an independent risk factor for ischemic heart disease in men? The Quebec cardiovascular study. JACC 1998; 31:519-525.
15. Seed M, Hoppichler F, Reaveley D, McCarthy S, Thompson G R, Boerwinkle E, Utermann G. Relation of serum lipoprotein (a) concentration and apolippprotein(a) phenotype to coronary heart disease in patients with familial hypercholesterolemia. NEJM 1990; 322:1494-1499.
16. Cardoso-Saldana G, De La Pena-Diaz A, Zamora-Gonzalez J, Gomez-Ortega R, Posadas-Romero C, Izaguirre-Avila R, Malvido-Miranda E, Morales-Anduaga M E, Angles-Cano E. Ethnicity and lipoprotein(a) polymorphism in Native Mexican populations. Ann Hum Biol. 2006 March-April; 33(2):202-12.
17. Wu H D, Berglund L, Dimayuga C, Jones J, Sciacca R R, Di Tullio M R, Homma S. High lipoprotein(a) levels and small apolipoprotein(a) sizes are associated with endothelial dysfunction in a multiethnic cohort. J Am Coll Cardiol. 2004 May 19; 43(10):1828-33.
18. Kraft H G, Li ngenhel A, Pang R W, Delport R, Trommsdorff M, Vermaak H, Janus E D, Utermann G. Frequency distributions of apolipoprotein(a) kringle IV repeat alleles and their effects on lipoprotein(a) levels in Caucasian, Asian, and African populations: the distribution of null alleles is non-random. Eur J Hum Genet. 1996; 4(2):74-87.
19. Paultre F, Pearson T A, Weil H F, Tuck C H, Myerson M, Rubin J, Francis C K, Marx H F, Philbin E F, Reed R G, Berglund L. High levels of Lp(a) with a small apo(a) isoform are associated with coronary artery disease in African American and white men. Arterioscler Thromb Vasc Biol. 2000 December; 20(12):2619-24.
   Guideline #12: Homocysteine>14 umol/l

### Statements Regarding Diagnosis and Management are Suggestions Only and Medical Decision Making Must be Individualized to the Patient.

### Test

### Fasting Homocysteine

### Background

Elevated plasma homocysteine (Hcy) may reflect underlying disorders including renal dysfunction, thyroid dysfunction, and pernicious anemia. Elevated Hcy has also been associated with increased CAD risk and in patients with established CAD, plasma homocysteine levels>10 umol/l have been associated with a significant increase in mortality. Elevated homocysteine levels have also been associated with impaired vasoreactivity. Elevated blood Hcy levels reflect increased CHD risk.

Elevated homocysteine levels have been associated with increased restenosis following PCI and, a small study has indicated that reduction of homocysteine (baseline 11.4 umol/1−>7.5 umol/L) with folate, B12, and B6 following PCI is associated with a significant (p<0.03) 33% reduction in restenosis.

Treatment of elevated Hcy involves the use of folic acid, vitamin B6, vitamin B12, and betaine. However, recent investigations have cast doubt on the efficacy of treating Hcy and even the possibility of some harm. An investigation of treatment with folate, B6 and B12 in patients receiving bare metal stents that reduced blood Hcy levels from 12.2 umol/l to 9.0 umol/l reported no benefit and a higher percent requiring revascularization (15.8% versus 10.6%, p=0.05) in the vitamin treated group. The HOPE-2 investigation examined the effect of folate, B6 and B12 supplementation versus placebo in 5522 subjects for 5 years. Fewer patients on treatment suffered a stroke compared to placebo but more patients in the treatment group were hospitalized for unstable angina. There was no reported benefit in subjects in the higher Hcy distribution. The VISP trials investigated the effect of “low” or “high” dose B vitamin supplements in 3,680 subjects with Hcy of 13.4 umol/l at baseline and reported no effect on vascular outcomes over 2 years. However, in the subset with Hcy>14 umol/l at baseline, a 10% reduction in stroke and coronary events was reported. In the recent NORVIT study 3,749 post MI patients were randomized to combinations of 0.8 mg folate, 0.4 mg B12, 40 mg B6 or placebo. Baseline Hcy were ˜13 umol/l and reduced to ˜9.5 umol/l (−27%) in the active arms. No cardiovascular benefit was reported in the group receiving folate+B 12+B6, a trend to increase risk (RR 1.22) was reported. Subjects with baseline Hcy>13 umol/l had an increased risk when treated with combination B vitamin therapy compared to those with lower baseline Hcy.

### Fasting Homocysteine

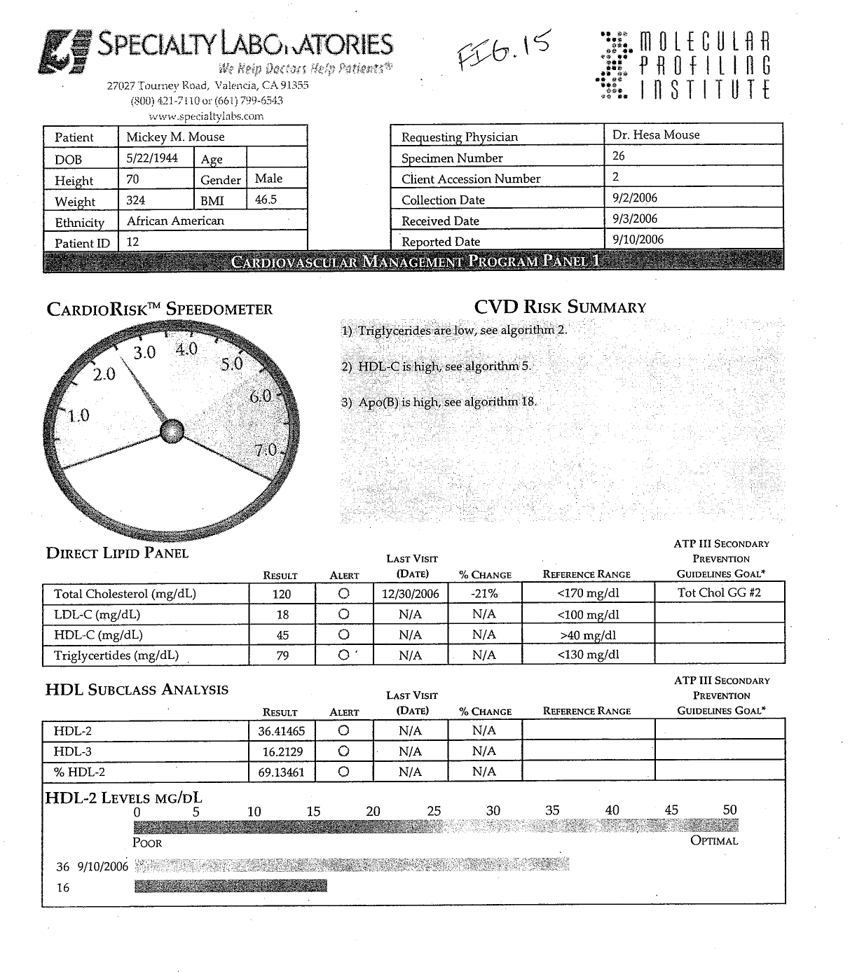

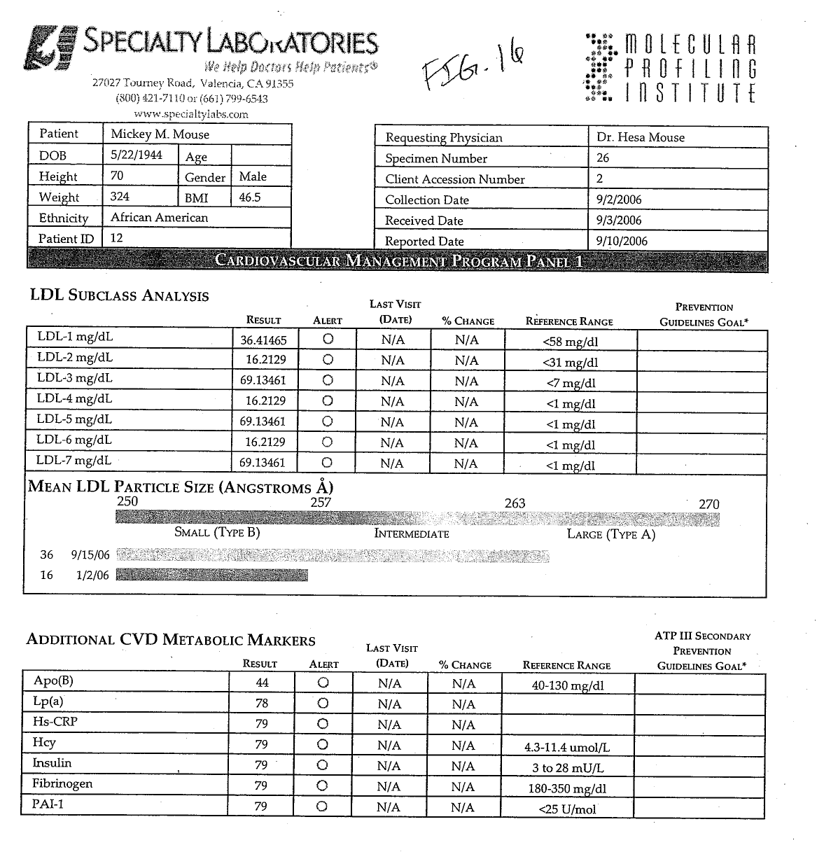

FIG. 12 illustrates a flowchart for carrying out a twelfth guideline in the system and method of the present invention for diagnosing and managing patients at risk for cardiovascular disease.

Result #1. Hcy<10 umol/l
Hcy not associated with increased CHD risk.
   Result #2. Hcy≧10 and ≧14 umol/l
Hcy associated with increased risk in a CHD population.
   Result #3. Hcy≧14 umol/l
Elevated Hcy associated with increase CHD risk and may reflect other metabolic disorders.
Consider screening for renal and thyroid dysfunction.
Consider screening for pernicious anemia.

### RELATED READING INCORPORATED BY REFERENCE

1. Graham I M, Daly L E, Refsum H M, Robinson K, Brattstrom L E, Ueland P M, Palma-Reis R J, Boers G H J, Sheahan R G, Israelsson B, Uiterwaal C S, Meleady R, McMaster D, Verhoef P, Witteman J, Rubba P, Bellet H, Wautrecht J C, de Valk H W, Sales A C, Parrot-Roulaud F M, Tan K S, Higgins I, Garcon D, Medrano M J, Candito M, Evans A E, Andria G. Plasma Homocysteine as a Risk Factor for Vascular Disease. The European Concerted Action Project. JAMA 1997; 277:1775-1781.
2. Nygard O, Nordrehaug E, Refsum H, Ueland P M, Farstad M, Vollset S E. Plasma homocysteine levels and mortality in patients with coronary artery disease. NEJM 1997; 337:230-236.
3. Zhao X Q, Kosinski A J S, Malinow M R, Superko H R, King S B. Association of total plasma homocysteine levels and 8-Year Mortality Following PTCA or CABG in EAST. Circulation 2000; 102; II-699.
4. Schnyder G, Roffi M, Flammer Y, Pin R, Hess O M. Association of plasma homocysteine with restenosis after percutaneous coronary angioplasty. Eut Heart J 2002; 23:726-733.
5. Schnyder G, Roffi M, Flammer Y, Pin R, Hess O M. Effect of homocysteine-lowering therapy with folic acid, vitamin B12, and vitamin B6 on clinical outcome after percutaneous coronary intervention. JAMA 2002; 288:973-979.
6. Lonn E, Yusuf S, Arnold M J, et al. HOPE 2 Investigators. Homocysteijne lowering with folic acid and B vitamins in vascular disease. NEJM 2006; 354:1567-1577.
7. Bonaa K H, Njolstad I, Ueland P M, Schirmer H, Tverdal A, Steigen T, Wang H, Nordrehaug J E, Arnesen E, Rasmussen K; NORVIT Trial Investigators. Homocysteine lowering and cardiovascular events after acute myocardial infarction. N End J. Med. 2006 Apr. 13; 354(15):1578-88.
8. Lange H, Suryapranata H, Giuseppe D L, et al. Folate therapy and in-stent restenosis after coronary stenting. NEJM 2004; 350:2673-2681.
9. Toole J, Malinow M, Chambless L, et al. Lowering homocysteine in patients with ischemic stroke to prevent recurrent stroke, myocardial infarction and Death. JAMA 2004; 291:565-575.

### Guideline #13. PAI-1

### Statements Regarding Diagnosis and Management are Suggestions Only and Medical Decision Making Must be Individualized to the Patient.

### Test

Plasminogin Activator Inhibitor-1 (PAI-1)

### Background

Reduced endogenous fibrinolysis secondary to increased PAI-1 appears to exacerbate atherogenesis and is associated with increased CHD risk. This is also associated with elevated insulin levels. The PAI-1 4G/4G genotype is associated with increased MI risk due to increased PAI-1 blood levels. In the Caerphilly Study, a 13 year study of 2,398 men, multivariable analysis with conventional risk factors, PAI-1, fibrinogen, and D-dimer were significantly associated with increased CVD risk. Recent AHA/ACC guidelines recommend PAI-1 as a potentially clinically useful marker.

Niacin can reduce PAI-mRNA and PAI-1 synthesis by 72%. Niacin has been shown in tissue culture to reduce TNF-alpha induced rise in ICAM-1 levels by 66% to 89% (p<0.0001). Gemfibrozil has been reported to attenuate the insulin-induced augmentation of PAI-1 in tissue culture by 61% (p<0.01). Cerivastatin and simvastatin have been reported to reduce PAI-1 production in tissue culture. The effect of directly targeting elevated PAI-1 as a treatment goal has not been addressed as a primary hypothesis in clinical trials. Elevated PAI-1 can be considered as a component of the increased thrombosis potential associated with the metabolic syndrome.

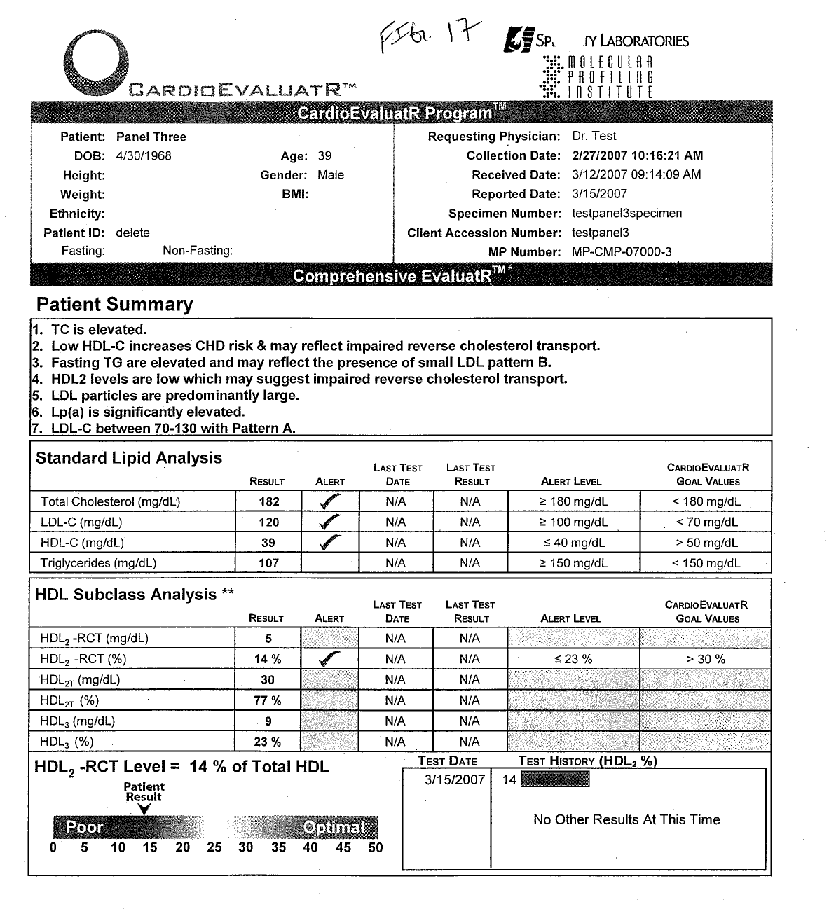

FIG. 13 illustrates a flowchart for carrying out a thirteenth guideline in the system and method of the present invention for diagnosing and managing patients at risk for cardiovascular disease.

### Result #1. PAI-≦xx

PAI-1 is not elevated. Goal achieved.

Result #2. PAI-1>xx≦xx
PAI-1 is moderately elevated and reflects moderate increase in CHD risk.
Consider testing for other factors that amplify the CHD risk associated with elevated PAI-1 including fibrinogen, factor VII, tissue-type plasminogen activator, insulin, and vonWillebrand factor.
If other factors reflect increased CHD risk, consider appropriate treatment with statin, niacin, fibrate, or a TZD if clinically appropriate.

### Result #3. PAI-1>xx

PAI-1 is elevated. CHD risk is increased.
Consider repeating the PAI-1 blood test to confirm the elevated value.
   Consider treatment with statin, niacin, fibrate, or a TZD if clinically appropriate.
Consider PAI-1 genotyping for family counseling.

### INCORPORATED REFERENCES

1. Tavaintharan S, Sivakumar M, Lim S C, Sum C F. Niacin affects cell adhesion molecules and plasminogen activator inhibitor-1 in HebG2 cells. Clin Chim Acta 2006; xx:xxx-xxx.
2. Brown S L, Sobel B E, Fujii S. Attenuation of the synthesis of plasminogen activator inhibitor type 1 by niacin. A potential link between lipid lowering and fibrinolysis. Circulation 1995; 92:767-772.
3. Nordt T K, Kornas K, Peter K, Fujii S, Sobel B E, Kubler W, Bode C. Attenuation by gemfibrozil of expression of plasminogen activator inhibitor type 1 induced by insulin and its precursors. Circulation 1997; 95:677-683.
4. Smith A, Patterson C, Yarnell J, Rumley A, Ben-Shlomo Y, Lowe G. Which hemostatic markers add to the predictive value of conventional risk factors for coronary heart disease and ischemic stroke? The Caerphilly Study. Circulation 2005; 112:3080-3087.
5. Choi B G, Vilahur G, Ibanez, Zafar M U, Rodriguez J, Badimon J J. Measures of thrombosis and fibrinolysis. Clin Lab Med 2006; 26:655-678.
   Guideline #14: Fasting Insulin>xx mg/dl

### Statements Regarding Diagnosis and Management are Suggestions Only and Medical Decision Making Must be Individualized to the Patient.

### Test

Fasting Insulin

### Background

The term “insulin resistance” is used to describe a metabolic state in which insulin action in the peripheral tissues is less efficient than normal which results in increased insulin secretion in order to maintain normal blood glucose levels. Moderate elevation in fasting insulin suggests the presence of insulin resistance and the insulin resistance syndrome.

A second definition of the “Metabolic syndrome” has been put forth by the World Health Organization (WHO) based on the European Group for the Study of Insulin Resistance and includes hyperinsulinemia or elevated fasting glycemia and >2 of the following: abdominal obesity, dyslipidemia, or hypertension (8). This alternative definition is useful since the ATP-III definition is less consistently predictive of CVD and all-cause mortality (9). Individuals can be insulin resistant without meeting the ATP criteria for the “Metabolic syndrome”.

This can be illustrated by the findings of the Quebec Cardiovascular Study in which the presence of small LDL, as the only metabolic abnormality, increased CV risk 3-fold, while the combination of small LDL and elevated apo B increased CV risk 6-fold, and the combined presence of small LDL, plus elevated apo B, plus elevated fasting insulin levels, increase CV risk 20-fold (25). The Malmo Heart Disease Prevention Project has reported that fasting and post glucose load insulin values were highly predictive of CV events in 5,484 healthy men followed for 19 years. In this project the mean fasting insulin in the group who did not suffer a CV event was 10.3 pU/L compared to 12.1 pU/L in those who did suffer a CV event.

In patients with established CHD, elevated post glucose load insulin values, but not glucose values, have been associated with PTCA and stent restenosis. In a population of “healthy” adults, the median fasting insulin is 10 pU/ml and the upper 1/3 starts at 12 pU/ml.
Consider an endocrinology consult and rule-out causes of hyperinsulinemia.
Consider the diagnosis of Metabolic Syndrome.
Avoid simple carbohydrates in the diet and consider a Mediteranean diet.
Reduce excess body fat.
Optimize physical activity.

### Repeat Fasting Triglycerides

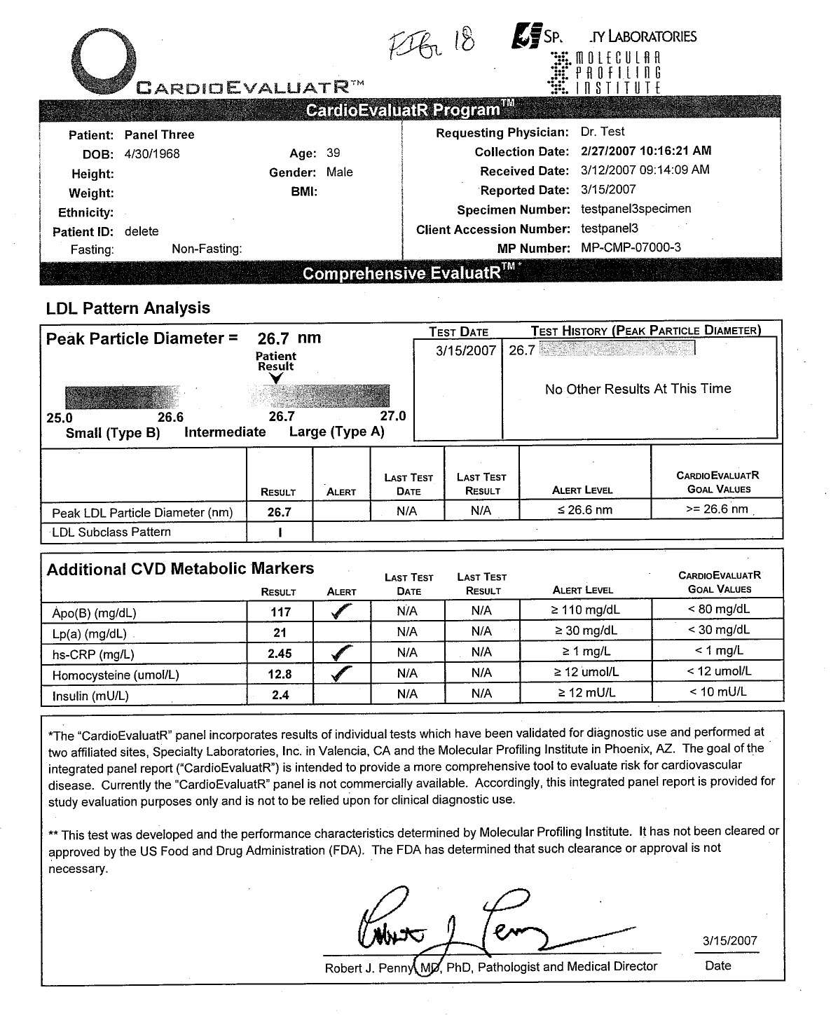

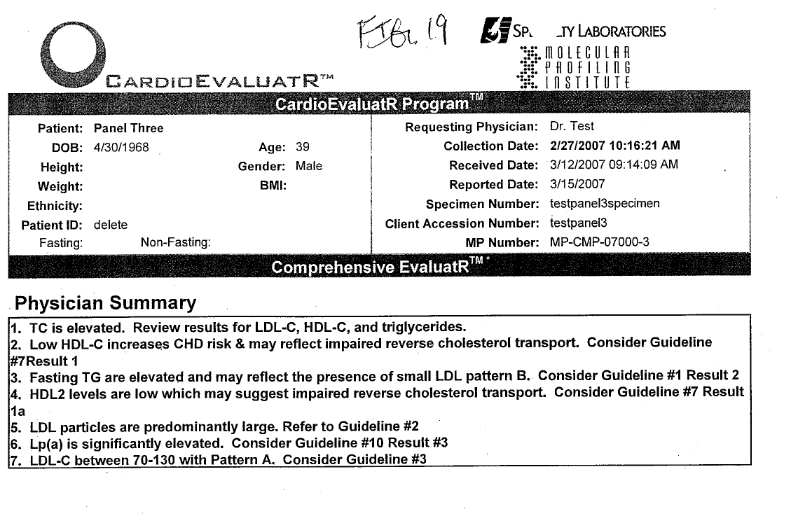

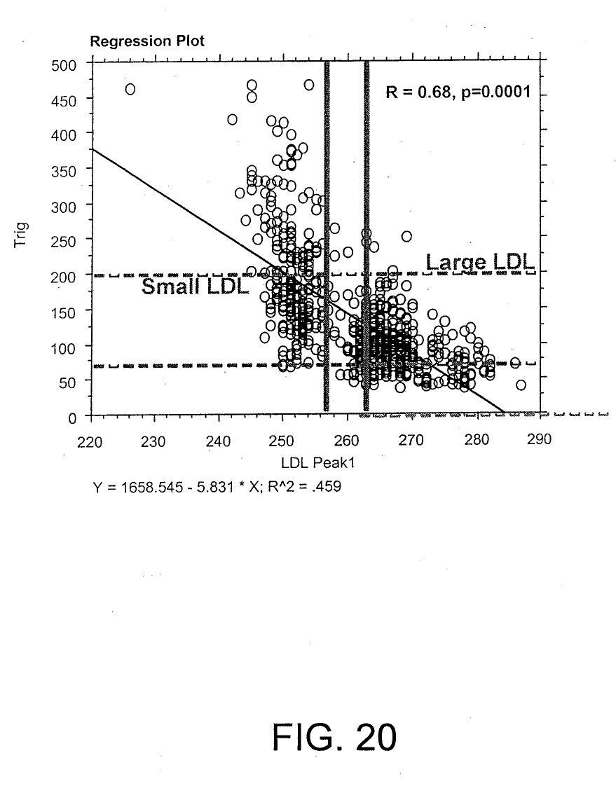

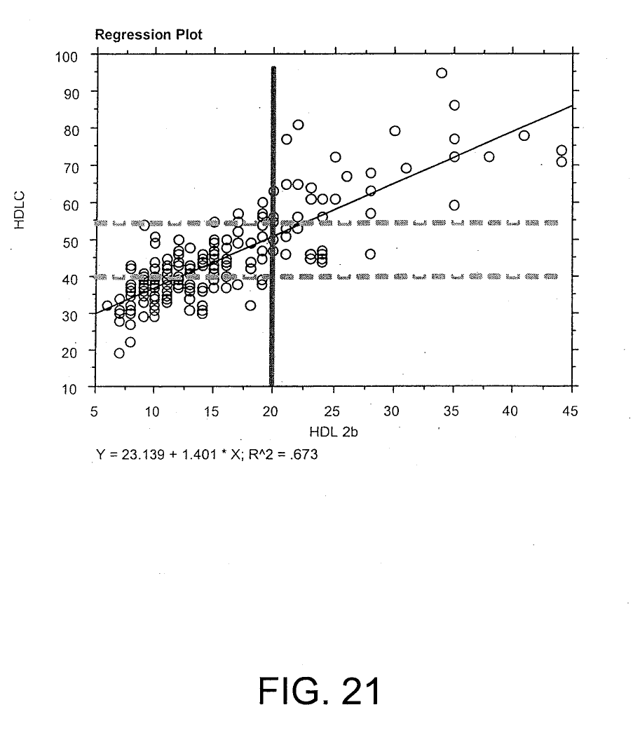

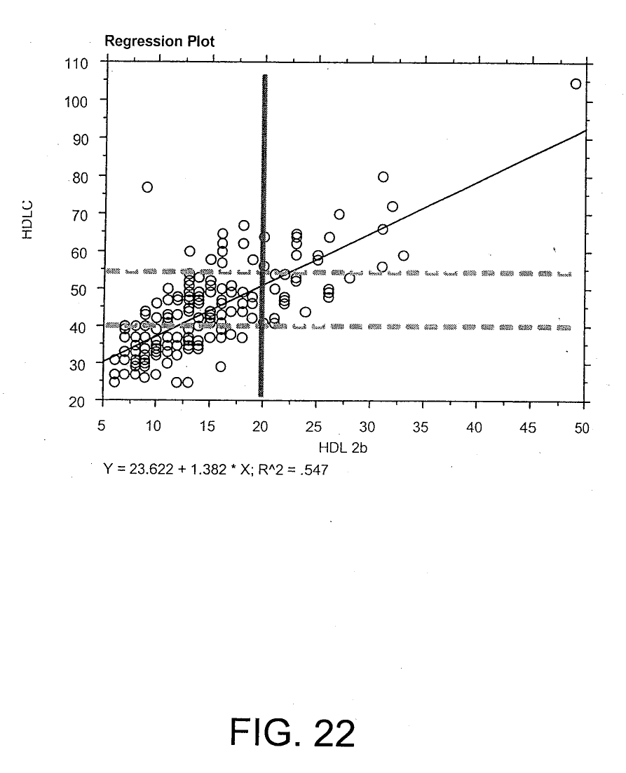

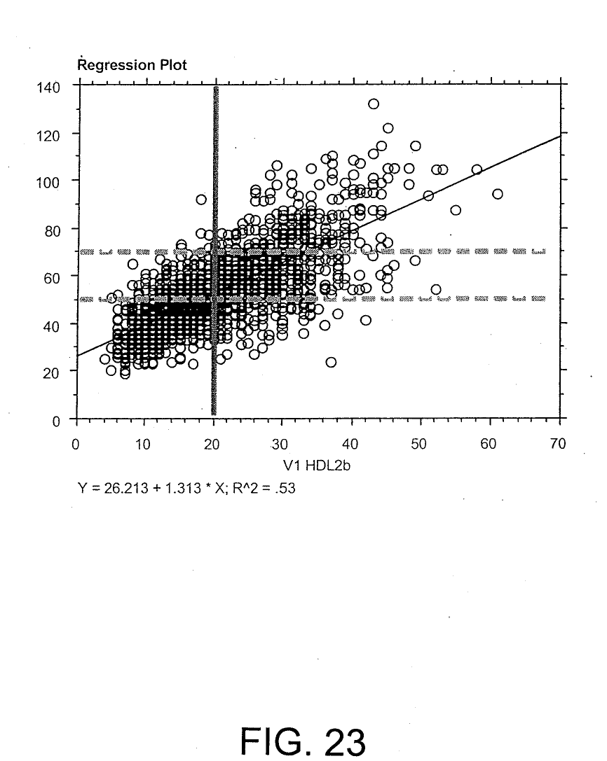

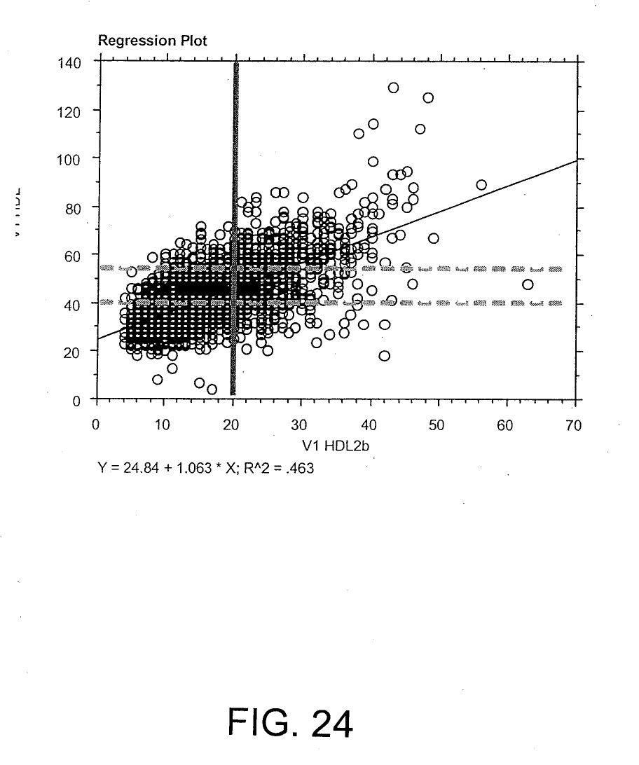

FIG. 14 illustrates a flowchart for carrying out a fourteenth guideline in the system and method of the present invention for diagnosing and managing patients at risk for cardiovascular disease.

Result #1. Fasting Insulin≦10 pU/ml
Fasting Insulin not elevated.
Consider post glucose load insulin testing if clinical suspicion exists.
   Result #2, Fasting Insulin≧10<12 pU/ml

Fasting Insulin moderately elevated.

Consider:
Glucose tolerance test
Reduce simple carbohydrates in the diet
Optimize exercise
Optimize body weight
Repeat fasting insulin after lifestyle adjustment
   Result #3. Fasting Insulin≧12 pU/ml

Fasting Insulin is elevated.

Consider:
Endocrinology consult
Glucose tolerance test
Reduce simple carbohydrates in the diet
High fiber diet (50 g/d)
Optimize exercise
Optimize body weight
Repeat fasting insulin after lifestyle adjustment
Thiazolodinediones
Metformin
Screen for other components of the Metabolic Syndrome

### RELATED READING INCORPORATED BY REFERENCE

1. Balkau B, Charles M A. Comment on the provisional report from the WHO consultation: European Group for the Study of Insulin Resistance (EGIR). Diabet med 1999; 16:44-443.
2. Lakka H M, Laaksonen D E, Niskanen L K, et al. The metabolic syndrome and total and cardiovascular disease mortality in middle-aged men. JAMA 2002; 288:2709-716.
3. Nilsson P. et al. The Malmo Preventive Project J of Internal Med 2003; 253:136-145.
4. Lamarche B, Tchernof A, Mauriege P, Cantin B, Dagenais G R, Lupien P J, Despres J P. Fasting insulin and apolipoprotein B levels and low-density lipoprotein particle size as risk factors for ischemic heart disease. JAMA 1998; 279:1955-1961.
5. Takagi T, Yoshida K, Akasaka T, Kaji S, Kawamoto T, Honda Y, Yamamuro A, Hozumi T, Morioka S. Hyperinsulinemia during oral glucose tolerance test is associated with increased neointimal tissue proliferation after coronary stent implantation in nondiabetic patients. J Am Coll Cardiol 2000; 36:731-738.
6. Nishimoto Y, Miyazaki Y, Toki Y, Murakami R, Shinoda M, Fukushima A, Kanayama H. Enhanced secretion of insulin plays a role in the development of atherosclerosis and restenosis of coronary arteries: elective percutaneous transluminal coronary angioplasty in patients with effort angina. J Am Coll Cardiol. 1998 Nov. 15; 32(6): 1624-9.
7. Reaven G M, Ida Chen Y D, Jeppesen J, Maheux P, Krauss R M. Insulin resistance and hyperinsulinemia in individuals with small, dense low density lipoprotein particles. J Clin Invest 1993; 92:141-146.
8. Reaven G. Metabolic Syndrome. Circulation 2002; 106:286-288.
9. Chandalia M, Garg A, Lutjohann D, von Bergmann K, Grudy S M, Brinkley L J. Beneficial effects of high dietary fiber intake in patients with type 2 diabetes mellitus. NEJM 2000; 342:1392-1398.
10. Despres J P, Lamarche B, Mauriege P, Cantin B, Dagenais G R, Moorjani S, Lupien P J. Hyperinsulinemia as an independent risk factor for ischemic heart disease. NEJM 1996; 334:952-957.
11. Ratner R E. An update on the Diabetes Prevention Program. Endocr Pract.
2006 January-February; 12 Suppl 1:20-4.
12. Pfutzner A, Schneider C A, Forst T. Pioglitazone: an antidiabetic drug with cardiovascular therapeutic effects. Expert Rev Cardiovasc Ther. 2006; 4:445-59.

We claim:

1. A method for diagnosing and managing individuals at risk for cardiovascular disease comprising the steps of:
determining an individual's fasting triglyceride (TG) level;
testing the individual for small low-density lipoprotein (LDL) depending on the individual's TG level;
providing disease management options based on the presence or absence of small LDL;
determining the individual's low-density lipoprotein cholesterol (LDL-C) level; and
providing disease management options based on the individual's LDL-C levels and the presence or absence of small LDL in the individual.

2. The method of claim 1 where the individual is tested for small LDL if the individual's TG level is greater than 70 mg/dl and less than 200 mg/dl and the presence of small LDL is presumed to be present in the individual if the individual's TG level is greater than or equal to 200 mg/dl.

3. The method of claim 2 wherein one or more of the following disease management options are performed if small LDL is present in the individual: loss of excess body fat by exercise and calorie restriction, avoidance of simple carbohydrates in the diet, avoidance of selective and nonselective beta blocker drugs if possible, confirmation of normal thyroid function, introduction of one or more of niacin, fibrates, and fish oil, high-density lipoprotein (HDL) subclass testing if high-density lipoprotein cholesterol (HDL-C) is normal, and testing first degree relatives for cardiovascular disease.

4. The method of claim 2 wherein one or more of the following disease management options are performed if LDL-C is within a range of 70 to 130 mg/dl and small LDL is present: avoidance of simple carbohydrates in the diet, reduction of body fat by exercise and calorie restriction, introduction of one or more of nicotinic acid, fibrates, statin and fish oil, and test of apolipoprotein B (Apo B) level as measurement of LDL particle number.

5. The method of claim 4 further comprising the step of performing a standard lipid profile test.

6. The method of claim 4 wherein management of the individual's risk for cardiovascular disease is achieved when the individual's LDL-C is less than 70 mg/dl and the individual's TG is less than 70 mg/dl.

7. The method of claim 5 further comprising the step of performing an LDL subclass test when the individual's LDL-C is less than 70 mg/dl and the individual's TG is greater than or equal to 70 mg/dl.

8. The method of claim 7 wherein management of the individual's risk for cardiovascular disease is achieved when the LDL subclass test results show an absence of small LDL in the individual.

9. The method of claim 7 further comprising at least one of increasing nicotinic acid to the individual, supplementing fibrate to the individual, and supplementing fish oil when the LDL subclass test results show a presence of small LDL in the individual.

10. The method of claim 5 further comprising at least one of increasing a statin dose to the individual and adding a dose of resin or ezetemibe to the individual if the individual's LDL-C is greater than or equal to 70 mg/dl and the individual's TG is greater than 70 mg/dl.

11. The method of claim 5 further comprising the step of performing an LDL subclass test when the individual's LDL-C is greater than 70 mg/dl and the individual's TG is greater than or equal to 70 mg/dl.

12. The method of claim 11 further comprising at least one of increasing nicotinic acid to the individual, increasing a statin dose to the individual, adding a dose of resin or ezetemibe to the individual, and supplementing fibrate to the individual when the LDL subclass test results show a presence of small LDL in the individual.

13. The method of claim 11 further comprising at least one of increasing a statin dose to the individual and adding a dose of resin or ezetemibe to the individual when the LDL subclass test results show an absence of small LDL in the individual.

14. The method of claim 2 wherein one or more of the following disease management options are performed if LDL-C is greater than or equal to 130 mg/dl and small LDL is absent: avoidance of simple carbohydrates in the diet, reduction of body fat by exercise and calorie restriction, consideration of familial combined hyperlipidemia (FCH) as diagnosis, introduction of one or more of nicotinic acid, fibrates, statin and fish oil, test of apolipoprotein B (Apo B) level as measurement of LDL particle number, and screening of first degree relatives for cardiovascular disease.

15. The method of claim 14 further comprising the step of performing a standard lipid profile test.

16. The method of claim 14 wherein management of the individual's risk for cardiovascular disease is achieved when the individual's LDL-C is less than 70 mg/dl and the individual's TG is less than 100 mg/dl.

17. The method of claim 15 further comprising the step of performing an LDL subclass test when the individual's LDL-C is less than 70 mg/dl and the individual's TG is greater than or equal to 100 mg/dl.

18. The method of claim 17 wherein management of the individual's risk for cardiovascular disease is achieved when the LDL subclass test results show an absence of small LDL in the individual.

19. The method of claim 17 further comprising at least one of increasing nicotinic acid to the individual, supplementing fibrate to the individual, and supplementing fish oil when the LDL subclass test results show a presence of small LDL in the individual.

20. The method of claim 15 further comprising at least one of increasing a statin dose to the individual and adding a dose of resin or ezetemibe to the individual if the individual's LDL-C is greater than or equal to 70 mg/dl and the individual's TG is greater than 100 mg/dl.

21. The method of claim 15 further comprising the step of performing an LDL subclass test when the individual's LDL-C is greater than or equal to 70 mg/dl and the individual's TG is greater than or equal to 100 mg/dl.

22. The method of claim 21 further comprising at least one of increasing nicotinic acid to the individual, increasing a statin dose to the individual, adding a dose of resin or ezetemibe to the individual, and supplementing fibrate to the individual when the LDL subclass test results show a presence of small LDL in the individual.

23. The method of claim 21 further comprising at least one of increasing a statin dose to the individual and adding a dose of resin or ezetemibe to the individual when the LDL subclass test results show an absence of small LDL in the individual.

24. The method of claim 2 wherein one or more of the following disease management options are performed if LDL-C is within a range of 70 to 130 mg/dl and small LDL is absent: an American Heart Association Phase X diet, a moderate dose of statin plus at least one of a dose of ezetemibe and a dose of bile acid binding resin, and test of apolipoprotein B (Apo B) level as measurement of LDL particle number.

25. The method of claim 24 further comprising the step of performing a standard lipid profile test.

26. The method of claim 24 wherein management of the individual's risk for cardiovascular disease is achieved when the individual's LDL-C is less than 70 mg/dl and the individual's TG has not increased.

27. The method of claim 25 further comprising the step of performing an LDL subclass test when the individual's LDL-C is less than 70 mg/dl and the individual's TG has increased.

28. The method of claim 27 wherein management of the individual's risk for cardiovascular disease is achieved when the LDL subclass test results show an absence of small LDL in the individual.

29. The method of claim 27 further comprising at least one of increasing nicotinic acid to the individual, supplementing fibrate to the individual, supplementing fish oil to the individual, and changing the individual's lifestyle when the LDL subclass test results show a presence of small LDL in the individual.

30. The method of claim 25 further comprising at least one of increasing a statin dose to the individual and adding a dose of resin or ezetemibe to the individual if the individual's LDL-C is greater than or equal to 70 mg/dl and the individual's TG has not increased.

31. The method of claim 25 further comprising the step of performing an LDL subclass test when the individual's LDL-C is greater than or equal to 70 mg/dl and the individual's TG has increased.

32. The method of claim 31 further comprising at least one of increasing nicotinic acid to the individual, supplementing fibrate to the individual, supplementing fish oil to the individual, and changing the individual's lifestyle when the LDL subclass test results show a presence of small LDL in the individual.

33. The method of claim 31 further comprising at least one of increasing a statin dose to the individual and adding a dose of resin or ezetemibe to the individual when the LDL subclass test results show an absence of small LDL in the individual.

34. The method of claim 2 wherein one or more of the following disease management options are performed if LDL-C is greater than 130 mg/dl and small LDL is absent: a low fat diet, an introduction of one or more of a statin, an ezetimibe, a resin, and nicotinic acid, and test of apolipoprotein B (Apo B) level as measurement of LDL particle number.

35. The method of claim 34 wherein management of the individual's risk for cardiovascular disease is achieved when the individual's LDL-C is less than 70 mg/dl and the individual's TG has not changed.

36. The method of claim 35 further comprising the step of performing an LDL subclass test when the individual's LDL-C is less than 70 mg/dl and the individual's TG has increased.

37. The method of claim 36 wherein management of the individual's risk for cardiovascular disease is achieved when the LDL subclass test results show an absence of small LDL in the individual.

38. The method of claim 36 further comprising at least one of increasing nicotinic acid to the individual, supplementing fibrate to the individual, and supplementing fish oil to the individual when the LDL subclass test results show a presence of small LDL in the individual.

39. The method of claim 34 further comprising at least one of increasing a statin dose to the individual and adding a dose of resin or ezetemibe to the individual if the individual's LDL-C is greater than or equal to 70 mg/dl and the individual's TG has not changed.

40. The method of claim 35 further comprising the step of performing an LDL subclass test when the individual's LDL-C is greater than 70 mg/dl and the individual's TG has increased.

41. The method of claim 40 further comprising at least one of adding nicotinic acid to the individual, increasing a dose of statin to the individual, supplementing the individual with at least one of a resin and an ezetimibe, and supplementing the individual with a fibrate when the LDL subclass test results show a presence of small LDL in the individual.

42. The method of claim 40 further comprising at least one of increasing a statin dose to the individual and adding a dose of resin or ezetemibe to the individual when the LDL subclass test results show an absence of small LDL in the individual.
